(19) United States
(12) Patent Application Publication
(10) Pub. No.: US 2025/0188050 A1
(43) Pub. Date: Jun. 12, 2025

(54) RECOMBINANT POLYPEPTIDES WITH BERBERINE BRIDGE ENZYME ACTIVITY USEFUL FOR THE BIOSYNTHESIS OF CANNABINOIDS

(71) Applicant: Invizyne Technologies, Inc., Monrovia, CA (US)

(72) Inventors: Tyler KORMAN, Sierra Madre, CA (US); John BILLINGSLEY, Los Angeles, CA (US); Mohammad HAYAT, Rolling Hills Estates, CA (US)

(73) Assignee: Invizyne Technologies, Inc., Monrovia, CA (US)

(21) Appl. No.: 18/726,859

(22) PCT Filed: Jan. 6, 2023
(86) PCT No.: PCT/US2023/060204

§ 371 Date: Jul. 5, 2024

Related U.S. Application Data
(60) Provisional application No. 63/297,561, filed on Jan. 7, 2022.

(51) Int. Cl. C07D 311/70; C12N 9/00; C12N 9/02; C12N 9/10; C12N 9/88; C12P 17/06

(52) U.S. Cl. CPC C07D 311/70; C12N 9/0004; C12N 9/1029; C12N 9/1085; C12N 9/88; C12N 9/93; C12P 17/06; C12Y 121/03003; C12Y 203/01206; C12Y 404/01026

(57) ABSTRACT

The application relates to recombinant polypeptides having berberine bridge enzyme (BBE) activity that are derived from microorganisms, such as Phytohabitans sufuscus, Streptomyces sp. AJS327, Streptomyces varsoviensis, Actinomadura pelletieri, Streptomyces flaveolus, Streptomyces sp. Ru71. or Streptomyces sp. CNH287, and the use of these recombinant polypeptides in compositions and methods for the oxidative cyclization of prenylated compounds, such as CBGA, in carrying out the biosynthesis, including cell-free biosynthesis, of cyclized cannabinoid compounds, such as CBCA, THCA, and CBDA.

### CROSS REFERENCE TO RELATED APPLICATIONS

This application claims priority benefit to U.S. Provisional Application No. 63/297,561, filed Jan. 7, 2022, which is hereby incorporated by reference herein.

### FIELD

The present disclosure relates to recombinant polypeptides with berberine bridge enzymes (BBE) activity and the use of these polypeptides in compositions and methods for biosynthesis of cannabinoids.

### REFERENCE TO SEQUENCE LISTING

The official copy of the Sequence Listing is submitted concurrently with the specification as an WIPO Standard ST.26 formatted XML file with file name “15041-003PV1.xml”, a creation date of Jan. 4, 2023, and a size of 445,667 bytes. This Sequence Listing filed electronically via USPTO EFS-Web is part of the specification and is incorporated in its entirety by reference herein. This sequence listing corresponds to the ST.25 formatted sequence listing with a file name of “15041-003PV1_SeqList_ST25.txt”, a creation date of Dec. 14, 2021, and a size of 445,667 bytes, that was filed with the priority U.S. Provisional Application No. 63/297,561 on Jan. 7, 2022.

### BACKGROUND

Cannabinoids are a large, well-known class of bioactive plant-derived compounds that regulate the cannabinoid receptors (CB1 and CB2) of the human endocannabinoid system. Cannabinoids are promising pharmacological agents with over 100 ongoing clinical trials investigating their therapeutic benefits as antiemetics, anticonvulsants, analgesics and antidepressants. Further, three cannabinoid therapies have been FDA approved to treat chemotherapy induced nausea, MS spasticity and seizures associated with severe epilepsy.

Although the plant, Cannabis sativa, is known to make over 100 different cannabinoid compounds, the best known and most studied cannabinoids include tetrahydrocannabidiolic acid (THCA), tetrahydrocannabidivarinic acid (THCVA), cannabidiolic acid (CBDA), cannabidivarinic acid (CBDVA), and their decarboxylated analogs (e.g., THC, THCV, CBD, CBDV). Of the cannabinoids made by the plant, nearly all are derived from the precursors cannabigerolic acid (CBGA) or cannabigerovarinic acid (CBGVA). CBGA and CBGVA are derived through the enzymatic prenylation of the polyketides olivetolic acid (OA) or divarinic acid (DA) respectively with geranyl pyrophosphate (GPP) through the action of the membrane protein GPP: OA transferase (GOT). Following formation of CBGA (or CBGVA), the plant uses a specific soluble berberine-bridge enzyme (BBE) such as THCA synthase (THCAS), CBDA synthase (CBDAS), or CBCA synthase (CBCAS) to cyclize the prenylated aromatic compound into the final downstream cannabinoids THCA, CBDA, or CBCA, respectively.

BBEs are a large family of FAD dependent enzymes that catalyze a variety of oxidations including oxidative cyclizations. Currently, however, the only BBEs known to catalyze the oxidative cyclization of CBGA to the downstream cannabinoids, THCA, CBDA, or CBCA, are the THCAS, CBDAS, and CBCAS enzymes from Cannabis sativa. These synthases from C. sativa cannot be expressed in a soluble form in E. coli systems for purification and use in in vitro cannabinoid biosynthesis. Accordingly, there is a need for alternative recombinant polypeptides with BBE activity.

### SUMMARY

The present disclosure relates generally to recombinant polypeptides having berberine bridge enzyme (BBE) activity that are derived from bacterial host organisms (not from C. sativa), such as Phytohabitans suffuscus, Streptomyces sp. AJS327, Streptomyces varsoviensis, Actinomadura pelletieri, Streptomyces flaveolus, Streptomyces sp. Ru71, or Streptomyces sp. CNH287, and the use of these recombinant polypeptides in compositions and methods for the oxidative cyclization of prenylated compounds, such as CBGA, in carrying out the biosynthesis of the cyclized product compounds, such as the cyclized cannabinoids, CBCA, THCA, and/or CBDA. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure also provides a method for preparing a recombinant polypeptide having BBE activity of the present disclosure, wherein the method comprises culturing a host cell comprising a polynucleotide or an expression vector encoding the polypeptide and isolating the polypeptide from the cultured host cell.

In at least one embodiment, the present disclosure also provides a method for preparing a compound of structural formula (I)

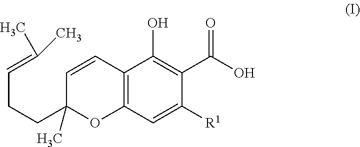

wherein, R¹ is C1-C7 alkyl,
   the method comprising contacting under suitable reactions conditions a compound of structural formula (II)

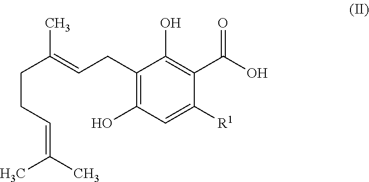

wherein, R¹ is C1-C7 alkyl,
   and a recombinant polypeptide having BBE activity of the present disclosure; optionally, wherein the recombinant polypeptide comprises an amino acid sequence of at least 80%, at least 90%, at least 95%, at least 97%, at least 98%, or at least 99% identity to a sequence selected from SEQ ID NO: 8, 2, 4, 6, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

In at least one embodiment of the method for preparing a compound of structural formula (I): (a) the compound of structural formula (I) is cannabichromenic acid (CBCA) and the compound of structural formula (II) is cannabigerolic acid (CBGA); (b) the compound of structural formula (I) is cannabichromevarinic acid (CBCVA) and the compound of structural formula (II) is cannabigerovarinic acid (CBGVA); or (c) the compound of structural formula (I) is cannabichromephorolic acid (CBCPA) and the compound of structural formula (II) is cannabigerophorolic acid (CBGPA).

In at least one embodiment of the method for preparing a compound of structural formula (I), the suitable reaction conditions comprise: (a) a cell-free solution; (b) a substrate compound of structural formula (II), 0.1 M buffer pH 8.0, and the recombinant polypeptide at 298 K for at least 1 hour; (c) a substrate compound of structural formula (II) of at least about 0.6 g/L, at least about 1.2 g/L, 2 g/L, 6 g/L, 12 g/L, 18 g/L, 24 g/L, 30 g/L or even greater; (d) a recombinant polypeptide concentration of about 0.1 g/L to about 5 g/L, or even lower concentration; (e) a pH of about 4.0 to about 11.0, or about 5.0 to 10.0; and/or (f) a buffer solution of about 0.05 M Tris-CI pH 8.0 to about 0.5 M Tris-CI pH 8.0.

In at least one embodiment, the present disclosure provides a composition comprising: (a) a recombinant polypeptide having BBE activity of the present disclosure; and (b) one or more enzymes that produce a substrate for the recombinant polypeptide. In at least one embodiment of the composition, the recombinant polypeptide having BBE activity comprises an amino acid sequence of at least 80%, at least 90%, at least 95%, at least 97%, at least 98%, or at least 99% identity to a sequence selected from SEQ ID NO: 8, 2, 4, 6, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

In at least one embodiment, the composition comprising the recombinant polypeptide having BBE activity and the one or more enzymes that produce a substrate is in a cell-free solution.

In at least one embodiment of the composition, the one or more enzymes that produce a substrate comprise an enzyme that converts a cannabinoid precursor compound (e.g., OA, DA, PA) to a prenylated aromatic compound (e.g., CBGA, CBGVA, CBGPA); optionally, wherein the prenylated aromatic compound is selected from: CBGA, CBGVA, CBGPA, and a combination thereof. In at least one embodiment, the composition comprises an enzyme that is prenyltransferase (e.g., NphB); optionally, the composition comprising a prenyltransferase further comprises a substrate for a prenyltransferase (e.g., OA, DA, PA).

In at least one embodiment of the composition, the one or more enzymes comprise: (a) a plurality of enzymes that convert isoprenol or prenol to geranylpyrophosphate (GPP); and (b) enzymes that convert a cannabinoid precursor compound (e.g., OA, DA, PA) to a prenylated aromatic compound (e.g., CBGA, CBGVA, CBGPA).

In at least one embodiment of the composition, the one or more enzymes comprise: (a) a plurality of enzymes that convert isoprenol or prenol to geranylpyrophosphate (GPP); (b) enzymes that convert a cannabinoid precursor (e.g., OA, DA, PA) to a prenylated aromatic compound (e.g., CBGA, CBGVA, CBGPA); (c) enzymes that convert malonate and acetyl-CoA to malonyl-CoA; and/or (d) enzymes that convert ADP and/or AMP to ATP; optionally, wherein the enzymes that convert ADP and/or AMP to ATP also convert acetyl-phosphate to acetic acid.

In at least one embodiment of the composition, the one or more enzymes comprise: (i) Acyl activating enzyme 3 (AAE3); (ii) Olivetol synthase (OLS); (iii) Olivetolic acid cyclase (OAC); and/or (iv) Prenyltransferase (NphB).

In at least one embodiment of the composition, the one or more enzymes comprise: (i) Acetyl-phosphate transferase (PTA); (ii) Malonate decarboxylase alpha subunit (mdcA); (iii) Acyl activating enzyme 3 (AAE3); (iv) Olivetol synthase (OLS); (v) Olivetolic acid cyclase (OAC); (vi) Prenyltransferase (NphB); (vii) Hydroxyethylthiazole kinase (ThiM); (viii) Isopentenyl kinase (IPK); (ix) Isopentyl diphosphate isomerase (IDI); (x) Diphosphomevalonate decarboxylase alpha subunit (MDCa); and/or (xi) Geranyl-PP synthase (GPPS) or Farnesyl-PP synthase mutant S82F (FPPS S82F).

In at least one embodiment, the present disclosure provides a recombinant polypeptide having berberine bridge enzyme (BBE) activity comprising an amino acid sequence of at least 80%, at least 90%, at least 95%, at least 97%, at least 98%, or at least 99% identity to SEQ ID NO: 2 and amino acid residue differences as compared to SEQ ID NO: 2 at one or more positions selected from T325, F156, L238, L283, T325, and G340; optionally, wherein the amino acid residue differences are selected from: T325G, F156Y, L238S, L283S, and G3401.

In at least one embodiment, the recombinant polypeptide having BBE activity further comprises amino acid residue differences as compared to SEQ ID NO: 2 at one or more positions selected from M101, A171, N267, L269, 1271, V323, E370, A398, N400, H402, D404, and/or T438; optionally, wherein the amino acid residue differences are selected from: M101A, A171Y, N267V, L269M, 1271H, V323Y, E370M, A398E, N400W, H402T, D404S, and/or T438Y

In at least one embodiment of the recombinant polypeptide having BBE activity, the polypeptide comprises a set of at least two amino residue differences selected from: M101A/T438Y; M101A/L269M/1271H; A171Y/A398E; L269M/1271H; L269M/1271H/T438Y; 1271H/T438Y; L283S/V323Y; E370M/T438Y; N400W/T438Y; N400W/H402T/D404S; N400W/H402T/D404S/T438Y; H402T/T438Y; and/or D404S/T438Y

In at least one embodiment of the recombinant polypeptide having BBE activity comprises an amino acid sequence of at least 80%, at least 90%, at least 95%, at least 97%, at least 98%, or at least 99% identity to a sequence selected from the group consisting of SEQ ID NO: 8, 2, 4, 6, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

In at least one embodiment of the recombinant polypeptide having BBE activity, the BBE activity of the polypeptide as compared to a polypeptide consisting of SEQ ID NO: 2 is increased at least 1.2-fold, at least 1.5-fold, at least 2-fold, at least 5-fold, or more; optionally, wherein the BBE activity is measured as the rate of the conversion of CBGA to CBCA. In at least one embodiment, the BBE activity is measured as the rate of conversion of the substrate cannabigerolic acid (CBGA) to the products, THCA and/or CBCA, under reaction conditions of 2.5 mM OA, 5 mM GPP, 5 mM MgCl₂, 50 mM Tris at pH 8.0 and 298 K.

In at least one embodiment of the recombinant polypeptide having BBE activity, the BBE activity of the polypeptide as compared to a polypeptide consisting of SEQ ID NO: 2 exhibits increased product selectivity that increased at least 1.2-fold, at least 1.5-fold, at least 2-fold, at least 5-fold, or more; optionally, wherein the product selectivity is measured as the rate of conversion of the substrate, CBGA to the product, CBCA, as compared to the conversion of the substrate, CBGA to the product, THCA.

In at least one embodiment, the present disclosure also provides a polynucleotide encoding a recombinant polypeptide having BBE activity of the present disclosure. In at least one embodiment, the polynucleotide encoding the polypeptide comprises a sequence of at least 80%, at least 90%, at least 95%, at least 97%, at least 98%, or at least 99% identity to a sequence selected from the group consisting of SEQ ID NO: 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, and 81.

In at least one embodiment, the present disclosure also provides an expression vector comprising a polynucleotide encoding recombinant polypeptide having BBE activity of the present disclosure; optionally, the expression vector comprises a control sequence.

In at least one embodiment, the present disclosure also provides a host cell comprising a polynucleotide or an expression vector comprising a polynucleotide, wherein the polynucleotide encodes recombinant polypeptide having BBE activity of the present disclosure.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts a schematic overview of steps, molecular inputs/outputs, and enzymes involved in the biosynthesis of various cannabinoid compounds relevant to the recombinant BBE polypeptide compositions and methods of the present disclosure and their use in the biocatalytic preparation of cannabinoids. The general pathway depicted is used in both cell-based (in vivo) and cell-free (in vitro) systems.

FIG. 2 depicts chemical structure of the exemplary cannabinoids THCA, THCVA, THCPA, CBCA, CBCVA, and CBCPA, that can be biosynthesized using the recombinant BBE polypeptides of the present disclosure.

FIG. 3 depicts a presumed mechanistic scheme for FAD-dependent quinone methide (QM) formation of CBGA catalyzed by the cannabinoid synthases from C. sativa, such as THCA synthase, CBDA synthase, or CBCA synthase. The bacterial BBE, Clz9, has been shown to catalyze a step of QM formation. The stereochemistry and/or isomerization of the resulting QM in the active site dictates the specific cyclization reaction that the highly reactive QM intermediate undergoes. Unlike the synthases from C. sativa, the active site of Clz9 and other bacterial BBE homologs only allows for CBCA formation.

FIG. 4 depicts a structural model of the Clz9 active site and the location of mutations described in the present disclosure. Sites in blue have a beneficial effect on enzyme activity when mutated to the corresponding residue from THCAS of C. sativa. Site L283 (grey) can only be mutated together with a V323Y mutation to avoid a loss of activity.

FIG. 5 depicts alignments of wild-type Clz9 amino acid sequence (SEQ ID NO: 2) with four variants, Clz9M1, Clz9M2, Clz9M3, and Clz9Mut34.

FIG. 6 depicts results from cyclization reactions catalyzed by wild-type Clz9 and the Clz9 variants as described in Example 1.

FIG. 7 depicts representative BBE gene clusters, from various bacterial source organisms, that contain polyketide synthase (PKSs) and/or prenyltransferase enzymes.

FIGS. 8A, 8B, 8C, 8D, and 8E depict results from cyclization reactions catalyzed at pH 7.5 by BBE homologs of Clz9 from Phytohabitans suffuscus (PsBBE) (FIG. 8B), Streptomyces varsoviensis (SvBBE) (FIG. 8C), Cannabis sativa THCAS (FIG. 8D), and Streptomyces sp. AJS327 (SAJBBE) (FIG. 8E), carried out as described in Example 2. Standards for the substrate compound CBGA, and the product compounds THCA and CBCA, are shown in FIG. 8A.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### Definitions

“Cannabinoid” refers to a compound that acts on cannabinoid receptor and is intended to include the endocannabinoid compounds that are produced naturally in animals, the phytocannabinoid compounds produced naturally in cannabis plants, and the synthetic cannabinoids compounds. Cannabinoids as referenced in the present disclosure include, but are not limited to, the exemplary naturally occurring and synthetic cannabinoid product compounds shown below in Table 1.

TABLE 1
Exemplary cannabinoid product compounds

| | Abbrev. | |
|---|---|---|
| Compound Name | Name | Chemical Structure |
| cannabigerolic acid | CBGA | 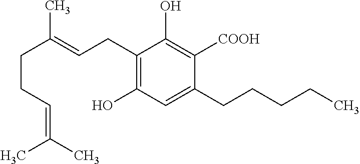 [SMILES: CCCCCc1cc(O)c(C/C=C(\C)CCC=C(C)C)c(O)c1C(=O)O] |
| cannabigerol | CBG | 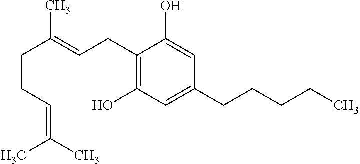 [SMILES: CCCCCc1cc(O)c(C/C=C(\C)CCC=C(C)C)c(O)c1] |
| Δ⁹-tetrahydrocannabinolic acid | Δ⁹-THCA | 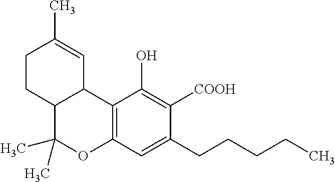 [SMILES: CCCCCc1cc2c(c(O)c1C(=O)O)C1C=C(C)CCC1C(C)(C)O2] |
| Δ⁹-tetrahydrocannabinol | Δ⁹-THC | 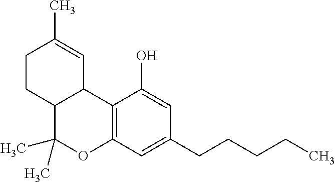 [SMILES: CCCCCc1cc(O)c2c(c1)OC(C)(C)C1CCC(C)=CC21] |
| Δ⁸-tetrahydrocannabinolic acid | Δ⁸-THCA | 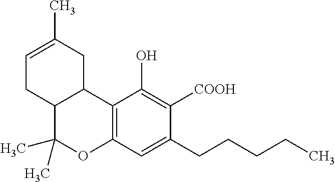 [SMILES: CCCCCc1cc2c(c(O)c1C(=O)O)C1CC(C)=CCC1C(C)(C)O2] |
| Δ⁸-tetrahydrocannabinol | Δ⁸-THC | 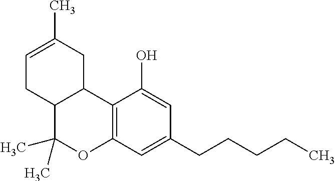 [SMILES: CCCCCc1cc(O)c2c(c1)OC(C)(C)C1CC=C(C)CC21] |
| cannabidiolic acid | CBDA | 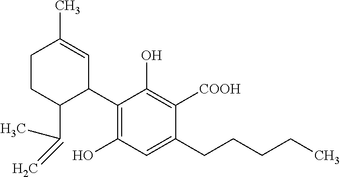 [SMILES: C=C(C)C1CCC(C)=CC1c1c(O)cc(CCCCC)c(C(=O)O)c1O] |
| cannabidiol | CBD | 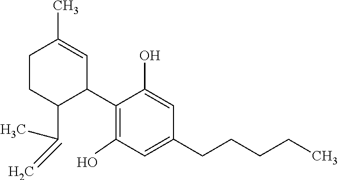 [SMILES: C=C(C)C1CCC(C)=CC1c1c(O)cc(CCCCC)cc1O] |
| cannabichromenic acid | CBCA | 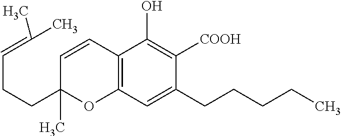 [SMILES: CCCCCc1cc2c(c(O)c1C(=O)O)C=CC(C)(CCC=C(C)C)O2] |
| cannabichromenic acid (B isomer) | CBCA-B | 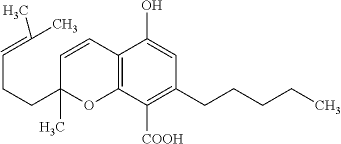 [SMILES: CCCCCc1cc(O)c2c(c1C(=O)O)OC(C)(CCC=C(C)C)C=C2] |
| cannabichromene | CBC | 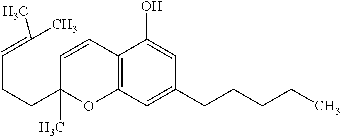 [SMILES: CCCCCc1cc(O)c2c(c1)OC(C)(CCC=C(C)C)C=C2] |
| cannabinolic acid | CBNA | 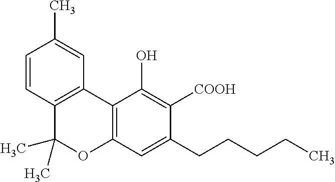 [SMILES: CCCCCc1cc2c(c(O)c1C(=O)O)-c1cc(C)ccc1C(C)(C)O2] |
| cannabinol | CBN | 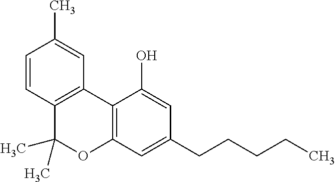 [SMILES: CCCCCc1cc(O)c2c(c1)OC(C)(C)c1ccc(C)cc1-2] |
| cannabigerovarinic acid | CBGVA | 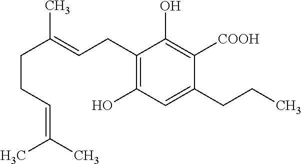 [SMILES: CCCc1cc(O)c(C/C=C(\C)CCC=C(C)C)c(O)c1C(=O)O] |
| cannabigerovarin | CBGV | 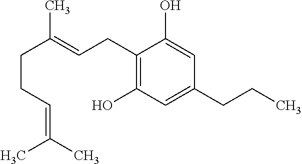 [SMILES: CCCc1cc(O)c(C/C=C(\C)CCC=C(C)C)c(O)c1] |
| cannabidivarinic acid | CBDVA | 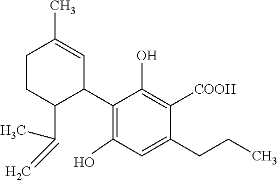 [SMILES: C=C(C)C1CCC(C)=CC1c1c(O)cc(CCC)c(C(=O)O)c1O] |
| cannabidivarin | CBDV | 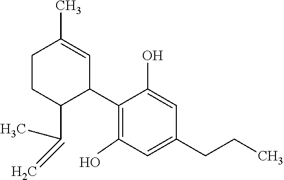 [SMILES: C=C(C)C1CCC(C)=CC1c1c(O)cc(CCC)cc1O] |
| Δ⁹-tetrahydrocannabivarinic acid | Δ⁹- THCVA | 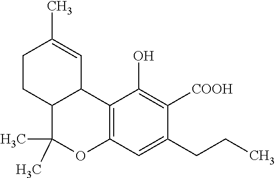 [SMILES: CCCc1cc2c(c(O)c1C(=O)O)C1C=C(C)CCC1C(C)(C)O2] |
| Δ⁹-tetrahydrocannabivarin | Δ⁹-THCV | 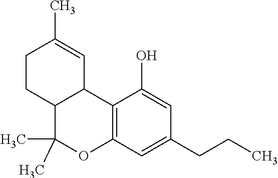 [SMILES: CCCc1cc(O)c2c(c1)OC(C)(C)C1CCC(C)=CC21] |
| cannabichromevarinic acid | CBCVA | 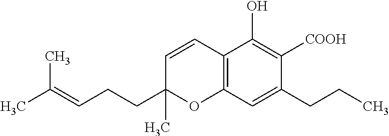 [SMILES: CCCc1cc2c(c(O)c1C(=O)O)C=CC(C)(CCC=C(C)C)O2] |
| cannabichromevarin | CBCV | 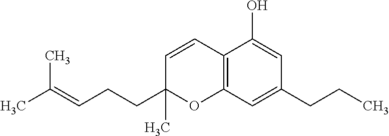 [SMILES: CCCc1cc(O)c2c(c1)OC(C)(CCC=C(C)C)C=C2] |
| cannabigerosphaerophorolic acid | CBGPA | 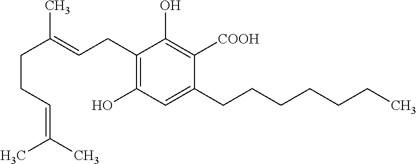 [SMILES: CCCCCCCc1cc(O)c(C/C=C(\C)CCC=C(C)C)c(O)c1C(=O)O] |
| cannabigerosphaerophorol | CBGP | 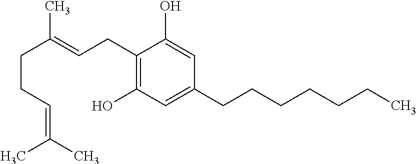 [SMILES: CCCCCCCc1cc(O)c(C/C=C(\C)CCC=C(C)C)c(O)c1] |
| cannabigerol-unsaturated- sphaerophorolic acid | CBGuPA | 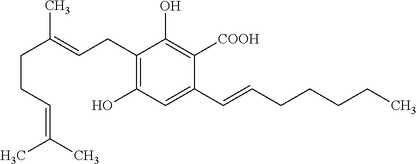 [SMILES: CCCCC/C=C/c1cc(O)c(C/C=C(\C)CCC=C(C)C)c(O)c1C(=O)O] |
| cannabigerol-unsaturated- sphaerophorol | CBGuP | 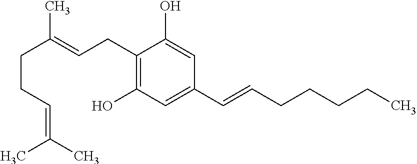 [SMILES: CCCCC/C=C/c1cc(O)c(C/C=C(\C)CCC=C(C)C)c(O)c1] |
| cannabidiphorolic acid | CBDPA | 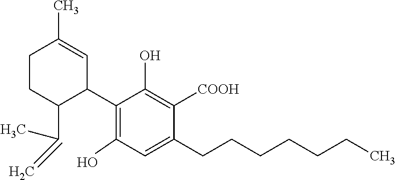 [SMILES: C=C(C)C1CCC(C)=CC1c1c(O)cc(CCCCCCC)c(C(=O)O)c1O] |
| cannabidiphorol | CBDP | 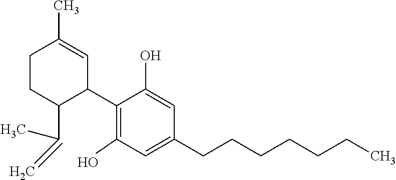 [SMILES: C=C(C)C1CCC(C)=CC1c1c(O)cc(CCCCCCC)cc1O] |
| Δ⁹-tetrahydrocannabiphorolic acid | Δ⁹-THCPA | 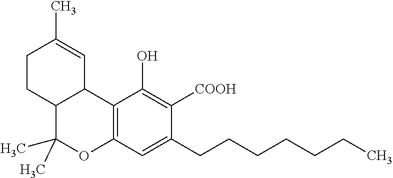 [SMILES: CCCCCCCc1cc2c(c(O)c1C(=O)O)C1C=C(C)CCC1C(C)(C)O2] |
| Δ⁹- tetrahydrocannabiphorol | Δ⁹-THCP | 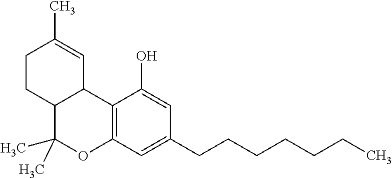 [SMILES: CCCCCCCc1cc(O)c2c(c1)OC(C)(C)C1CCC(C)=CC21] |
| cannabichromephorolic acid | CBCPA | 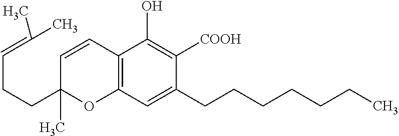 [SMILES: CCCCCCCc1cc2c(c(O)c1C(=O)O)C=CC(C)(CCC=C(C)C)O2] |
| cannabichromephorol | CBCP | 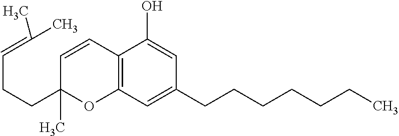 [SMILES: CCCCCCCc1cc(O)c2c(c1)OC(C)(CCC=C(C)C)C=C2] |
| cannabidibutolic acid | CBDBA | 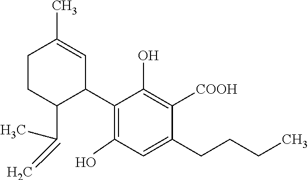 [SMILES: C=C(C)C1CCC(C)=CC1c1c(O)cc(CCCC)c(C(=O)O)c1O] |
| cannabidibutol | CBDB | 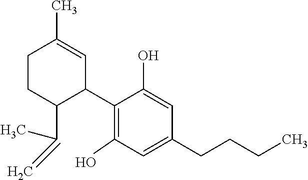 [SMILES: C=C(C)C1CCC(C)=CC1c1c(O)cc(CCCC)cc1O] |
| Δ⁹- tetrahydrocannabutolic acid | Δ⁹-THCBA | 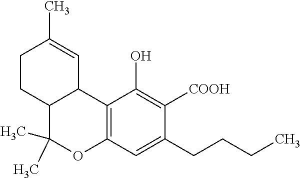 [SMILES: CCCCc1cc2c(c(O)c1C(=O)O)C1C=C(C)CCC1C(C)(C)O2] |
| Δ⁹- tetrahydrocannabutol | Δ⁹-THCB | 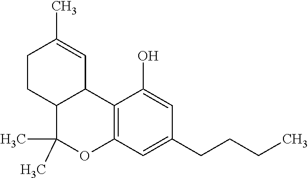 [SMILES: CCCCc1cc(O)c2c(c1)OC(C)(C)C1CCC(C)=CC21] |
| cannabicyclolic acid | CBLA | 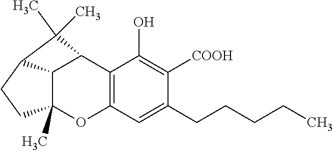 |
| cannabicyclol | CBL | 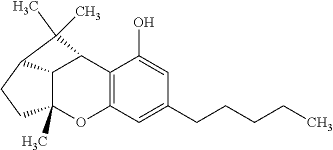 |
| cannabielsoinic acid | CBEA | 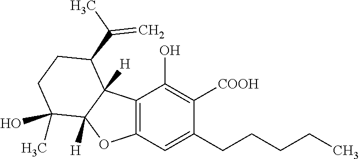 [SMILES: C=C(C)[C@@H]1CC[C@](C)(O)[C@H]2Oc3cc(CCCCC)c(C(=O)O)c(O)c3[C@@H]12] |
| cannabielsoin | CBE | 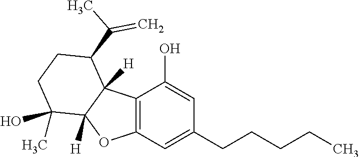 [SMILES: C=C(C)[C@@H]1CC[C@](C)(O)[C@H]2Oc3cc(CCCCC)cc(O)c3[C@@H]12] |
| cannabicitranic acid | CBTA | 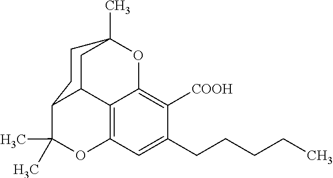 [SMILES: CCCCCc1cc2c3c(c1C(=O)O)OC1(C)CCC(C3C1)C(C)(C)O2] |
| cannabicitran | CBT | 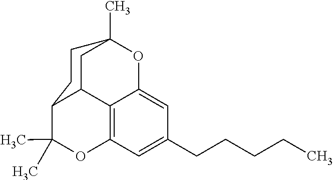 [SMILES: CCCCCc1cc2c3c(c1)OC(C)(C)C1CCC(C)(CC31)O2] |

“Conversion” as used herein refers to the enzymatic conversion of the substrate(s) to the corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Product” as used herein in the context of an enzyme mediated process refers to the compound or molecule resulting from the activity of the enzyme. In the context of the engineered polypeptides with BBE activity of the present disclosure, exemplary products include, but are not limited to, the cannabinoid compounds summarized in Table 1.

“Substrate” as used herein in the context of an enzyme mediated process refers to the compound or molecule acted on by the enzyme. In the context of the engineered polypeptides with BBE activity of the present disclosure, substrates acted on by the polypeptides can include a range of “cannabinoid” compounds, including but not limited to, the exemplary cannabinoid compounds CBGA, CBGVA, and CBGPA with varying alkyl carbon chain lengths as summarized in Table 1.

“Cannabinoid precursor compound” or “cannabinoid precursor substrate” as used herein refers a compound or molecule acted on by an enzyme in a biosynthetic step for producing a cannabinoid, e.g., polyketide substrate for a prenyltransferase that is prenylated to form a cannabinoid. Exemplary cannabinoid precursor compounds with a range of alkyl carbon lengths are provided in Table 2.

TABLE 2
Exemplary cannabinoid precursor substrate compounds

| | Abbrev. | |
|---|---|---|
| Compound Name | Name | Chemical Structure |
| 2,4-dihydroxy-6- methylbenzoic acid | | 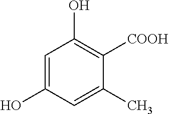 |
| 2-ethyl-4,6- dihydroxybenzoic acid | | 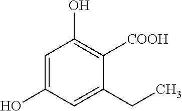 |
| Divarinic acid (2,4-dihydroxy-6- propylbenzoic acid) | DA | 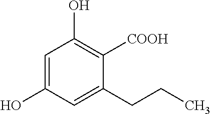 |
| Butolic acid (2-butyl-4,6- dihydroxybenzoic acid) | BA | 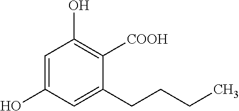 |
| Olivetolic acid (2,4-dihydroxy-6- pentylbenzoic acid) | OA | 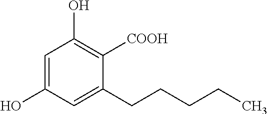 [SMILES: CCCCCc1cc(O)cc(O)c1C(=O)O] |
| 2-hexyl-4,6- dihydroxybenzoic acid | | 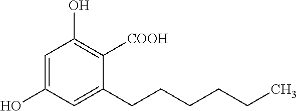 [SMILES: CCCCCCc1cc(O)cc(O)c1C(=O)O] |
| Sphaerophorolic acid (2-heptyl-4,6- dihydroxybenzoic acid) | PA | 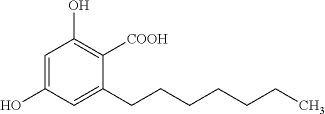 [SMILES: CCCCCCCc1cc(O)cc(O)c1C(=O)O] |
| Unsaturated sphaerophorolic acid | uPA | 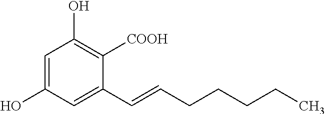 [SMILES: CCCCC/C=C/c1cc(O)cc(O)c1C(=O)O] |

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein interchangeably to refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo-and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristylation, ubiquitination, etc.). As used herein “protein” or “polypeptide” or “peptide” polymer can include D-and L-amino acids, and mixtures of D-and L-amino acids.

“Naturally-occurring” or “wild-type” as used herein refers to the form as found in nature. For example, a naturally occurring nucleic acid sequence is the sequence present in an organism that can be isolated from a source in nature and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from” as used herein refers to a nucleic acid having a sequence at least substantially identical to a sequence of found in naturally in an organism. For example, cDNA molecules prepared by reverse transcription of mRNA isolated from an organism, or nucleic acid molecules prepared synthetically to have a sequence at least substantially identical to, or which hybridizes to a sequence at least substantially identical to a nucleic sequence found in an organism.

“Coding sequence” refers to that portion of a nucleic acid (e.g., a gene) that encodes an amino acid sequence of a protein.

“Heterologous nucleic acid” as used herein refers to any polynucleotide that is introduced into a host cell by laboratory techniques and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; Mclnerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215:403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Substantial identity” or “substantially identical” refers to a polynucleotide or polypeptide sequence that has at least 70% sequence identity, at least 80% sequence identity, at least 85% sequence identity, at least 90% sequence identity, at least 95% sequence identity, or at least 99% sequence identity, as compared to a reference sequence over a comparison window of at least 20 nucleoside or amino acid residue positions, frequently over a window of at least 30-50positions, wherein the percentage of sequence identity is calculated by comparing the reference sequence to a sequence that includes deletions or additions which total 20 percent or less of the reference sequence over the window of comparison.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., cannabinoid, polynucleotide, polypeptide) is substantially separated from other compounds that naturally accompany it, e.g., protein, lipids, and polynucleotides. The term embraces nucleic acids which have been removed or purified from their naturally-occurring environment or expression system (e.g., host cell or in vitro synthesis).

“Substantially pure” refers to a composition in which a desired molecule is the predominant species present (i.e., on a molar or weight basis it is more abundant than any other individual macromolecular species in the composition) and is generally a substantially purified composition when the object species comprises at least about 50 percent of the macromolecular species present by mole or % weight.

“Recovered” as used herein in relation to an enzyme, protein, or cannabinoid compound, refers to a more or less pure form of the enzyme, protein, or cannabinoid.

Recombinant Polypeptides with BBE Activity

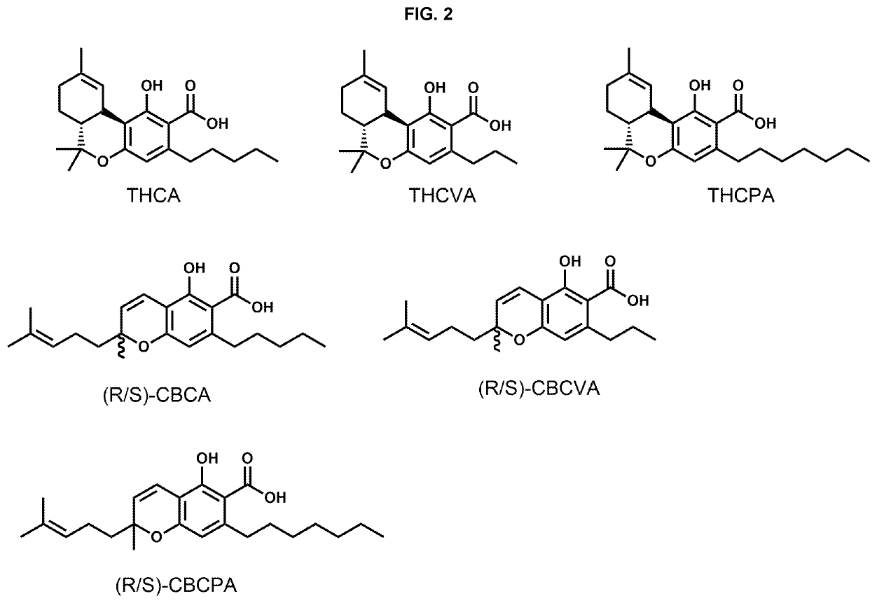

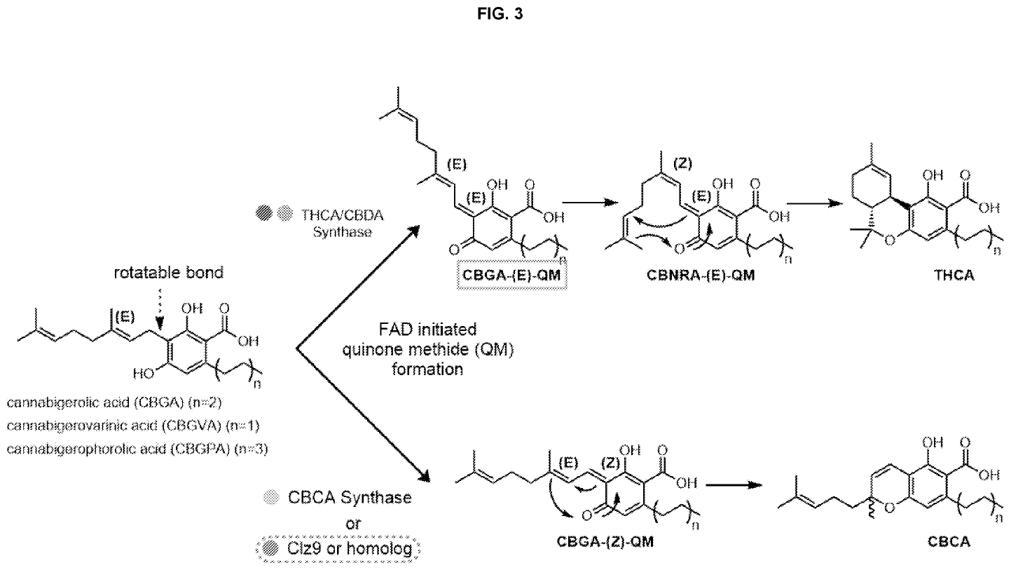

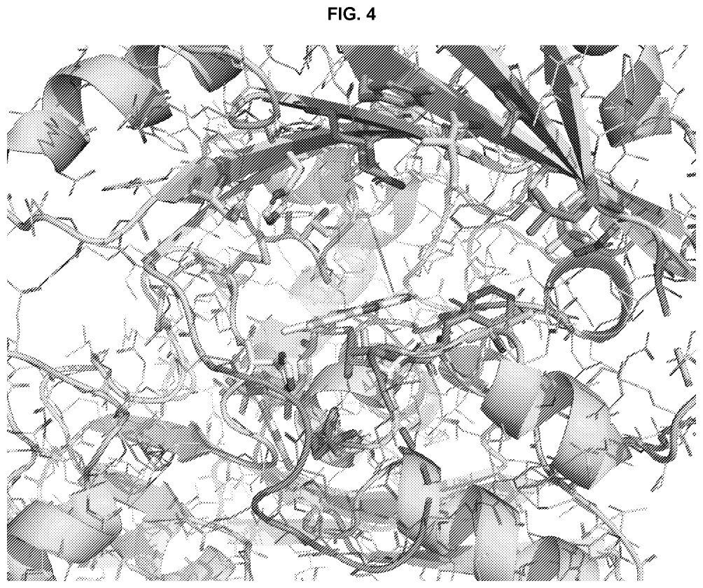

The present disclosure provides recombinant polypeptides having berberine bridge enzyme (BBE) activity and exhibiting increased selectivity relative to the CBCA synthase from C. sativa for the conversion of the prenylated aromatic cannabinoid substrate, CBGA to the cyclized cannabinoid product, CBCA, relative to the production of cannabinoid product, THCA. In particular, the recombinant polypeptides are capable of converting the prenylated aromatic cannabinoid substrate compounds, such as CBGA, CBGVA, or CBGPA, to the corresponding cannabinoid product compounds, CBCA, CBCVA, and CBCPA, respectively, with greater selectivity relative to the alternative cyclized cannabinoid product compounds, THCA, THCVA, and THCPA. FIG. 2 shows the differing structures of these cyclized cannabinoid products. Without intending to be bound by a mechanism, it is believed that the recombinant polypeptides having BBE activity of the present disclosure have active sites that favor the formation of the Z-form of the CBGA-quinone methide intermediate that leads to the selective formation of the CBCA product as depicted in FIG. 3. This mechanistic interpretation of the biocatalytic stereoselectivity is believed supported by the structure of the wild-type Clz9 active site, which is depicted in FIG. 4 and further by the site-directed mutation results provided by the Examples disclosed herein.

In one exemplary embodiment, the recombinant polypeptides having BBE activity of present disclosure are capable of converting the prenylated aromatic cannabinoid substrate, cannabigerolic acid (CBGA) (compound (2)) to the cyclized cannabinoid, cannabichromenic acid (CBCA) (compound (1)), as shown in Scheme 1, with increased selectivity relative to the production of the alternative, cyclized cannabinoid, THCA (compound (3), as shown in Scheme 2.

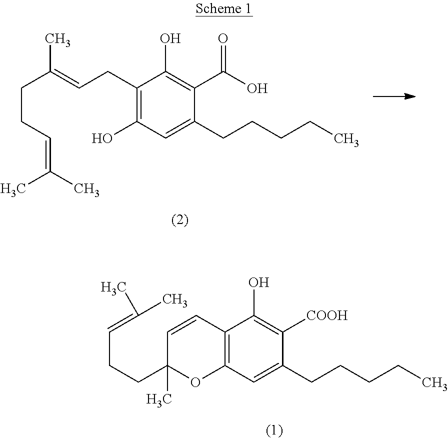 [SMILES: CCCCCc1cc(O)c(C/C=C(\C)CCC=C(C)C)c(O)c1C(=O)O ; CCCCCc1cc2c(c(O)c1C(=O)O)C=CC(C)(CCC=C(C)C)O2]

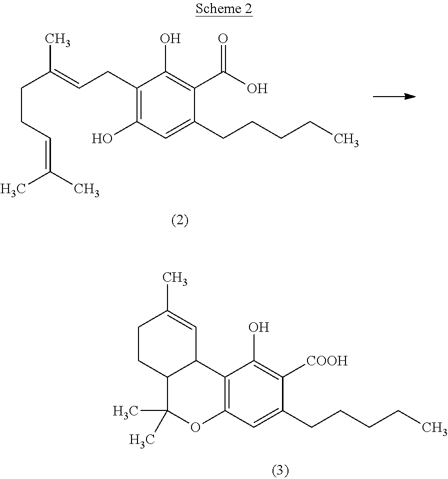

The recombinant polypeptides of the present disclosure which exhibit increased product selectivity and/or activity are derived from bacterial sources that exhibit very low sequence identity to the cyclized cannabinoid synthases CBCAS, THCAS, and CBDAS from C. sativa. Unlike these synthase enzymes from C. sativa, which are membrane bound and difficult to use in soluble cell-free systems, the bacterial homolog enzymes with BBE activity of the present disclosure have been found to be well-suited to expression in the E. coli and use in soluble cell-free systems, as described elsewhere herein including the Examples. A range of exemplary recombinant polypeptides with BBE activity derived from bacterial sources with the unexpected and surprising technical effect of increased CBCA selectivity are summarized in Table 3 below.

TABLE 3
Recombinant Polypeptides with BBE Activity from Bacterial Sources

| | | NT | AA | |
|---|---|---|---|---|
| Polypeptide | | SEQ | SEQ | |
| Name | | ID | ID | Relative |
| (Source) | AA Sequence | NO: | NO: | Activity¹ |
| Clz9 | MTADPSSERSDMNEADEVNEVDELSETGQTSGTKGKRP | 1 | 2 | ++ |
| (Streptomyces | FTGRVIGPADGEFDEARRVWNECFAARPKEIVYCADTR | | | |
| sp. CNH287) | DVVRALREVRQRGGPFRVRSGGHSMSGLSVLDDGTVLD | | | |
| | VSGLDDIQVSEDASTVTVGSGAHLGDIFRALWARGVTV | | | |
| | PAGFCPEIGIAGHVLGGGAGILVRSRGFLSDHLVALEM | | | |
| | VDSEGRIVVADHDSHHELLWASRGGGGGNFGIATSFTL | | | |
| | RTQPIGDVTLFTIAWDWDRGAEAIKAWQEWLATADGRI | | | |
| | NTLFIAYPQDQDMFAALGCFEGDAAELEPLIAPLVHAV | | | |
| | EPTEKVAETMPWIEALSFVETMQGEAMKATSVRAKGNL | | | |
| | SFVTEPLGDRAVEEIKKALAQAPSHRAEVVLYGLGGAV | | | |
| | AAKGRRETAFVHRDAPVALNYHTDWDDEAEDDLNFAWI | | | |
| | QNLRASVAAHTEGRGSYVNTIDLTVEHWLWDYYEENLP | | | |
| | RLMAVKKRYDPEDVFRHPQSIPVSLTEAEAAELGIPPH | | | |
| | IAEELRAARQLR | | | |
| SfBBE | MINRRSFLTGAGGVALATTALTGAAQAKGDAFGTRWSR | 3 | 4 | — |
| (Streptomyces | LADRLQGRLVLPSDADYQRAKQLYQVQFDATSPRAVAY | | | |
| flaveolus) | CVSAADVSACVRFAEAYGIPVAARSGGHSAGGYSTTEG | | | |
| | LVIDVSGLNSIVLGESSVRMGPGAQLVDIGDTLAPAGL | | | |
| | GISGGYCPTVAAGGFFQGGGMGLFTRSVGIASDKVNSA | | | |
| | QVVLADGRVVTASPRSHSDLFWALRGGGGGNFGIVTSY | | | |
| | DVTPSPLTDVAAINLVWTFDQSLDMLDGWTRWLPDAPW | | | |
| | TIGSGVNVTLTDAGPGTVPTASVFLGSVDTGAGFEAEI | | | |
| | ARLISLVGRPPVYQQKFTAPYRSVLMSLYQCSDLTAQQ | | | |
| | CHRADTSPDGRIQRPAFGAWRSRLFERTMPREGWAKAL | | | |
| | ETFNANRFAGQMRQLQISALGGRANTVARDATAYVHRD | | | |
| | SLFSVSYLTSNAAAPVADEAKAAAWEYVDDGFAVIDPY | | | |
| | SNGETYQNFIDPRLPDWRRSYYAENYARLARVKDKYDP | | | |
| | YRFFRFAQGVR | | | |
| SR71BBE | MVNRRSFLVGAGGAALAATGLASPAFARSSRGGKWHRL | 5 | 6 | ++ |
| (Streptomyces | SHHLGGRLVLPSDADYPTAKQLYLAQFDHIHPQAIAYC | | | |
| sp. Ru71) | TSPADVALCLRFAEYHDVPIAVRSGGNCAGGYSTKPDS | | | |
| | LVIDVSQMNQISIGSKSVKLGPGAALVDITDKLSRNGL | | | |
| | SIADGYCPSVAAGGFLSGGGAGPFTRAMGMATDSVTKA | | | |
| | QVVLADGRLVTASPHENPDLYWAVRGGGGGNFGVVTGY | | | |
| | QITPEPLTDLDTCTLFWPWDQGLEVFEGWSKWQADAPW | | | |
| | AIGSALLITLMDAAPGKVPVLSVFMGSVDTGGILAPEV | | | |
| | ERLISLVGRPPARRQPVKAPYKKIMMGFYNCDDKTVDE | | | |
| | CHRVSTWPDALLPRMEYGIWRSRLFSKPLPRGGWSDVL | | | |
| | QVFEDERWPGQMRQLQLVTMGGKANTVARDATAFVHRD | | | |
| | TIFSIAYLAANYTGPVSAETVTSAEHFADTGFNVIDPY | | | |
| | SNGETYLNFYDPRDPNWEKSYYAENYPRLMRIKDKYDP | | | |
| | YNIFNHDQSVR | | | |
| PsBBE | MSTAPVFGGRIVTRLDPSYEEARQVWNAVPDHRPLEIR | 7 | 8 | +++ |
| (Phytohabitans | YCRDAQDVAAALRDALARGLPFAARSGGHSAAALSVVP | | | |
| suffuscus) | DGVVIDVSPLNSISLDQDASQVTFGSGATLGAIYRTLW | | | |
| | ESGVTVPAGTCPNIGVAGHVLGGGLGILSRSRGPLVDH | | | |
| | LIGVTMVDARGELVTADEENHSDLLWACRGGGGGNYGI | | | |
| | ATSFTLRTQPIADVTLFNITWREDDLGPAVKAWQSWLA | | | |
| | DADHRINPFMMLYPREQDMVATVGVMEGTPDAFEPLVK | | | |
| | PLLDAVEPSEVVIEPMSFIQAVDTVEALQGEAAVAKSV | | | |
| | RALGSSAIIDRPLDDRALETLRRHLIDPPSHRSEVAIY | | | |
| | GMGGKIAAKGREETAFVHRTELMAFEYRTDWDEPADDE | | | |
| | KNVAWVRSVRQEMAEHTTGGTYTNTVDLGVENWLWGYY | | | |
| | EENLPRLMGVKHRYDPDGVFQHAHSVPLSLTEAEASRF | | | |
| | GIPEQIRRTLRAAGRLT | | | |
| SAJBBE | MATPSAFSGSVLTPGDDGFEAAQVTWNACYSSRPREVM | 9 | 10 | ++ |
| (Streptomyces | VCHDAASVAEAVRSVRERGLPFRVRSGGHSMCGLSNLD | | | |
| sp. AJS327) | DGVIIDLGGLGGVELTPDRQTVRIGGGARLADVYNTLW | | | |
| | DHRLTVPAGTCPRIGVGGHVLGGGMGVLSRSRGALVDH | | | |
| | LTALEMVDAEGRLLRVSEDENPDLFWACRGGGGGNFGI | | | |
| | VTAYELRPTPIDDVTIFTVSWTWSQLPDAVRAWQRWLG | | | |
| | SAESRINSFLSLFPQQQDMVVAFGVFDGPAADFRPLLA | | | |
| | PLTAEVAPEAEVVEEVPFIQAVDTVEALQGEAAAAEQV | | | |
| | RAQGSSAIIANPLNDEALATLQEFLTDPPSHRAEVAVY | | | |
| | GMGGVIGERERGDTAFVHRTGLMAFEYRTDWDSPEDDR | | | |
| | LNLDWVTRLRHAMAEHTTGAAYVNTIDLALENWLWAYY | | | |
| | EENLPRLMAVKRRYDPENVFHHPHSIPGSLTAEAARAH | | | |
| | GVPEATLKRLHDDGLLDGPLD | | | |
| SvBBE | MSGATSARFAGTVLAPGDPGFEDASRTWNACFASRPRE | 11 | 12 | ++ |
| (Streptomyces | VMVCHSTAEVADAVREVRERGVSFRVRSGGHSMAGLSS | | | |
| varsoviensis) | VEDGVVIDLGGLDAISLSADRSLVTVGGGTRLGDIYRE | | | |
| | LWRSGVTVPAGTCPRIGIGGHVLGGGMGVLSRSRGALV | | | |
| | DHLAALEFVDAEGRAYRVDENEHPDLFWACRGGGGGSY | | | |
| | GIATSYTLRTSPVDEVTLFTLSWNWERFAPAVRAWQSW | | | |
| | LASTDDRVNTFLSLFPRQQDLVAAFGVYDGPAAEFEEL | | | |
| | LRPLVAAVRPQDQVVEPMPFIQAVDTVELLQGEAAAAD | | | |
| | QVRAQGSSAIIGGPLDGAAIATLQQFLTDPPSHRSEVA | | | |
| | VYGMGGAIARPARTDTAFVHRTGLMAFEYRTDWDRSED | | | |
| | DAKNLAWIKALRHAMAPHTTGAAYVNTIDLALENWLWA | | | |
| | YYEENLPRLIEVKRRYDPGDVFHHPHSIPLTVTEADIA | | | |
| | RYGIPEAVAARLRAAGLTDRQ | | | |
| ApBBE | MISRRSFVAGVGGAAALAATAVPTPAHARPASRIRWDR | 13 | 14 | ++ |
| (Actinomadura | LRRHLRGRLVLPSDADYPKAKQLYQMEFDEIHPQAIAY | | | |
| pelletieri) | CTCTSDVSLCLRFAQDHDLHLAVRSGGHSHGGYSTTPG | | | |
| | LVIDVSRMNSVVPGEQSTSIGTGAQTVDITNALAPAGL | | | |
| | AVSTGYCPTVAAGGFLQGGGVGLLTRATGIASDKVRSA | | | |
| | RVVLADGRVVTASPSRHEDLYWALRGGGGGNFGIVTSY | | | |
| | EVTPTPLTHLATAILIWRYDQALDMLDGWTKWLEDAPW | | | |
| | TIGSGLNVQLEDSAPGKVPVASVLVGSTDTGPGFAAEV | | | |
| | ERLVSLVGHAPTFRRDEVEPYQKIMMGLFDCDDLTVDE | | | |
| | CHRSDTFPSGRLPRAAFGFERSRMFGRAMPRDGWAKTL | | | |
| | ALFDTERRARQTHQLQVTALGGRANTLSRTETAYVHRD | | | |
| | TRFMVTLYGWHREGPVSAEDKAVAGRWIDAAFNVIDPY | | | |
| | SNGETYQNFIDPRLTDWQRSYYAENYRRLKRIKHRYDP | | | |
| | HNVFRSPQSIR | | | |

¹Relative Activity corresponds to the activity of the wild-type or variant Clz9 in the cell-free conversion rate of CBGA to CBCA in 4 hours at 32° C. under the assay conditions described in Example 1. Under these conditions wild-type Clz9 converts ~50% of the input CBGA to CBCA. Relative rates are indicated as follows: + = <25% conversion at 4 hours; ++ = >50% conversion at 4 hours; +++ = >75% conversion at 4 hours; ++++ = 100% conversion at 4 hours; — = no conversion.

As shown in Table 3, certain naturally occurring recombinant polypeptides with BBE activity from source organisms other than C. sativa are capable of converting the prenylated aromatic cannabinoid, CBGA (compound (2) to the cyclized cannabinoid product, CBCA (compound (1)) with greater selectivity than the wild-type CBCAS from C. sativa. The recombinant polypeptide, Clz9 (SEQ ID NO: 2) from Streptomyces sp. CNH287, was further engineered by site-directed mutagenesis as described in the Examples, to provide recombinant engineered polypeptides that have amino acid residue differences relative to SEQ ID NO: 2 and which exhibit BBE activity with various unexpected technical effects, including altered selectivity and/or activity relative to the wild-type Clz9 in carrying out the conversion of CBGA (compound (2)) to CBCA (compound (1). These recombinant engineered variants of Clz9 of are summarized in Table 4 below.

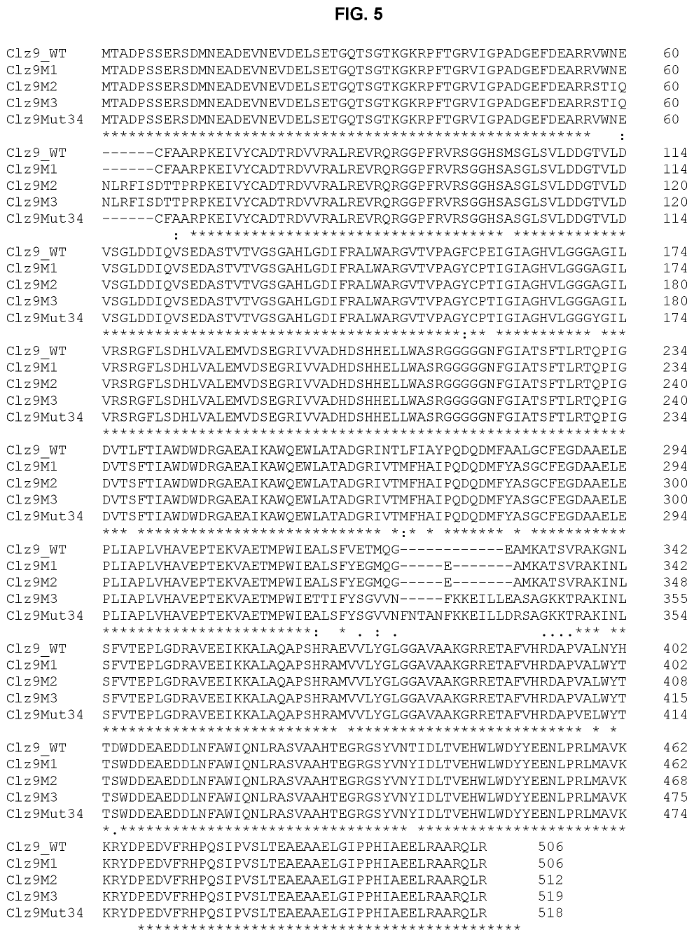

TABLE 4
Recombinant variants of Clz9

| | AA changes relative to wild- | NT SEQ | AA SEQ | Relative |
|---|---|---|---|---|
| Clz9 Variant | type Clz9 (SEQ ID NO: 2) | ID NO: | ID NO: | Activity¹ |
| Clz9M1 | (see alignment in FIG. 5) | 15 | 16 | − |
| Clz9M2 | (see alignment in FIG. 5) | 17 | 18 | − |
| Clz9M3 | (see alignment in FIG. 5) | 19 | 20 | − |
| Clz9M4 | M101A | 21 | 22 | ++++ |
| Clz9M5 | L238S | 23 | 24 | + |
| Clz9M6 | L269M | 25 | 26 | ++ |
| Clz9M7 | I271H | 27 | 28 | + |
| Clz9M8 | L269M/I271H | 29 | 30 | ++++ |
| Clz9M9 | L283S | 31 | 32 | − |
| Clz9M10 | T325G | 33 | 34 | +++ |
| Clz9M11 | E370M | 35 | 36 | ++ |
| Clz9M12 | N400W | 37 | 38 | ++ |
| Clz9M13 | H402T | 39 | 40 | +++ |
| Clz9M14 | D404S | 41 | 42 | ++++ |
| Clz9M16 | T438Y | 43 | 44 | ++++ |
| Clz9M17 | G340I | 45 | 46 | ++ |
| Clz9M19 | I271H/T438Y | 47 | 48 | ++ |
| Clz9M20 | M101A/T438Y | 49 | 50 | ++++ |
| Clz9M21 | M101A/L269M/I271H | 51 | 52 | |
| Clz9M22 | L269M/I271H/T438Y | 53 | 54 | ++ |
| Clz9M23 | E370M/T438Y | 55 | 56 | +++ |
| Clz9M24 | N400W/T438Y | 57 | 58 | +++ |
| Clz9M25 | H402T/T438Y | 59 | 60 | |
| Clz9M26 | D404S/T438Y | 61 | 62 | |
| Clz9M27 | F156Y | 63 | 64 | + |
| Clz9M28 | N267V | 65 | 66 | − |
| Clz9M29 | V323Y | 67 | 68 | ++ |
| Clz9M30 | L283S/V323Y | 69 | 70 | + |
| Clz9M31 | A171Y | 71 | 72 | |
| Clz9M32 | A398E | 73 | 74 | − |
| Clz9M33 | A171Y/A398E | 75 | 76 | − |
| Clz9Mut34 | (see alignment in FIG. 5) | 77 | 78 | |
| Clz9M35 | N400W/H402T/D404S | 79 | 80 | + |
| Clz9M36 | N400W/H402T/D404S/T438Y | 81 | 82 | + |

¹Relative Activity corresponds to the activity of the wild-type or variant Clz9 in the cell-free conversion rate of CBGA to CBCA in 4 hours at 32° C. under the assay conditions described in Example 1.
Under these conditions wild-type Clz9 converts ~50% of the input CBGA to CBCA. Relative rates are indicated as follows: + = <25% conversion at 4 hours; ++ = >50% conversion at 4 hours; +++ = >75% conversion at 4 hours; ++++ = 100% conversion at 4 hours; − = no conversion.

In at least one embodiment, the recombinant polypeptides having BBE activity and altered product selectivity and/or increased activity have one or more residue differences as compared to the reference recombinant polypeptide Clz9 of SEQ ID NO: 2. In at least one embodiment, the recombinant polypeptides have one or more residue differences at residue positions selected from M101, F156, A171, L238, N267, L269, 1271, L283, V323, T325, G340, E370, A398, N400, H402, D404, and/or T438.

It is to be understood that the residue differences from SEQ ID NO: 2 at residue positions associated with altered product selectivity and/or increased activity can be used in various combinations to form recombinant polypeptides having desirable enzymatic characteristics, for example combination of increased conversion rate, increased product yield, and/or improved utilization of substrate. Exemplary combinations are described herein. For example, the present disclosure provides a recombinant polypeptide having BBE activity and altered product selectivity and/or increased activity wherein, the polypeptide comprises an amino acid sequence of at least 80% identity to SEQ ID NO: 2, and amino acid residue differences as compared to SEQ ID NO: 2 at one or more positions selected from: M101, F156, A171, L238, N267, L269, 1271, L283, V323, T325, G340, E370, A398, N400, H402, D404, and/or T438. In at least one embodiment, the amino acid residue differences are selected from: M101A, F156Y, A171Y, L238S, N267V, L269M, 1271H, L283S, V323Y, T325G, G3401, E370M, A398E, N400W, H402T, D404S, and/or T438Y.

In at least one embodiment of the recombinant polypeptide having BBE activity and altered product selectivity and/or increased activity, it is contemplated that the polypeptide comprises an amino acid sequence with a specific set of amino residue differences relative to SEQ ID NO: 2. In at least one embodiment the set comprises at least two amino residue differences selected from: M101A/T438Y; M101A/L269M/1271H; A171Y/A398E; L269M/1271H; L269M/1271H/T438Y; 1271H/T438Y; L283S/V323Y; E370M/T438Y; N400W/T438Y; N400W/H402T/D404S; N400W/H402T/D404S/T438Y; H402T/T438Y; and/or D404S/T438Y.

Based on the correlation of recombinant polypeptide functional information provided herein with the sequence information provided in Tables 3, 4 and the accompanying Sequence Listing, one of ordinary skill can recognize that the present disclosure provides a range of recombinant polypeptides having BBE activity and altered product selectivity and/or increased activity, wherein the polypeptide comprises an amino acid sequence comprising one or more of the amino acid differences or sets of amino acid differences (relative to SEQ ID NO: 2) disclosed in any one of SEQ ID NO: 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82, and otherwise have at least 80%, at least 90%, at least 95%, at least 97%, at least 98%, or at least 99% identity to a sequence selected from the group consisting of SEQ ID NO: 2, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

Thus, in at least one embodiment, a recombinant polypeptide of the present disclosure having BBE activity and altered product selectivity and/or increased activity can have an amino acid sequence comprising one or more of the amino acid differences or sets of amino acid differences (relative to SEQ ID NO: 2) disclosed in any one of SEQ ID NO: 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82, and additionally have 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions.

In addition to the residue positions specified above, any of the engineered polypeptides BBE activity and altered product selectivity and/or increased activity disclosed herein can further comprise other residue differences relative to wild-type Clz9 polypeptide of SEQ ID NO: 2 at other residue positions. Residue differences at these other residue positions can provide for additional variations in the amino acid sequence without adversely affecting the ability of the recombinant polypeptide to carry out the desired biocatalytic conversion (e.g., conversion of compound (2) to compound (1)). In some embodiments, the recombinant polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40 residue differences at other amino acid residue positions as compared to SEQ ID NO: 2. In some embodiments, the number of differences can be 1, 2, 3, 4, 5, 6, 7, 8, 9, 10, 11, 12, 14, 15, 16, 18, 20, 22, 24, 26, 30, 35, and 40 residue differences at other residue positions. The residue difference at these other positions can include conservative changes or non-conservative changes. In some embodiments, the residue differences can comprise conservative substitutions and non-conservative substitutions as compared to the wild-type Clz9 polypeptide of SEQ ID NO: 2.

In some embodiments, the recombinant polypeptides of the disclosure can be in the form of fusion polypeptides in which the engineered polypeptides are fused to other polypeptides, such as, by way of example and not limitation, antibody tags (e.g., myc epitope), purification sequences (e.g., His tags for binding to metals), and cell localization signals (e.g., secretion signals). Thus, the recombinant polypeptides described herein can be used with or without fusions to other polypeptides. It is also contemplated that the recombinant polypeptides described herein are not restricted to the genetically encoded amino acids. In addition to the genetically encoded amino acids, the polypeptides described herein may be comprised, either in whole or in part, of naturally-occurring and/or synthetic non-encoded amino acids.

In another aspect, the present disclosure provides polynucleotides encoding the recombinant polypeptides having BBE activity and altered product selectivity and/or increased activity as described herein. The polynucleotides may be operatively linked to one or more heterologous regulatory sequences that control gene expression to create a recombinant polynucleotide capable of expressing the polypeptide. Expression constructs containing a heterologous polynucleotide encoding the recombinant polypeptide can be introduced into appropriate host cells to express the corresponding polypeptide. Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved transaminase enzymes disclosed herein. Thus, having identified a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein, including the amino acid sequences presented in Tables 3 and 4, and the Sequence Listing.

The codons can be selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. It is contemplated that all codons need not be replaced to optimize the codon usage of the recombinant polypeptide since the natural sequence will comprise preferred codons and because use of preferred codons may not be required for all amino acid residues. Consequently, codon optimized polynucleotides encoding the recombinant polypeptide may contain preferred codons at about 40%, 50%, 60%, 70%, 80%, or greater than 90% of codon positions of the full-length coding region.

In at least one embodiment, the polynucleotide encoding a recombinant polypeptide having BBE activity and altered product selectivity and/or increased activity comprises an amino acid sequence that is at least about 80%, 85%, 90%, 91%, 92%, 93%, 94%, 95%, 96%, 97%, 98%, 99%, or more identical to the wild-type Clz9 sequence of SEQ ID NO:2. In some embodiments, the polynucleotide encodes a recombinant polypeptide comprising an amino acid sequence that has the percent identity to SEQ ID NO: 2 as described above, and has one or more amino acid residue differences as compared to SEQ ID NO:2 described elsewhere herein (e.g., Table 4), for example at residue positions selected from M101, F156, A171, L238, N267, L269, 1271, L283, V323, T325, G340, E370, A398, N400, H402, D404, and/or T438. In at least one embodiment, the polynucleotide sequence comprises a sequence of at least 80%, at least 90%, at least 95%, at least 97%, at least 98%, or at least 99% identity to a sequence selected from the group consisting of SEQ ID NO: 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, and 81.

The present disclosure provides an expression vector comprising a polynucleotide encoding a recombinant polypeptide having BBE activity and altered product selectivity and/or increased activity, and one or more expression regulating regions such as a promoter, a terminator, a replication origin, or the like, depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the recombinant polypeptide at such sites. Alternatively, a polynucleotide sequence of the present disclosure may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression. The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated.

Furthermore, a single vector or plasmid or two or more vectors or plasmids which together contain the total DNA to be introduced into the genome of the host cell, or a transposon may be used. In at least one embodiment, the expression vector further comprises one or more selectable markers, which permit easy selection of transformed cells.

The present disclosure also provides host cell comprising a polynucleotide or expression vector encoding a recombinant polypeptide of the present disclosure, wherein the polynucleotide is operatively linked to one or more control sequences for expression of the polypeptide having BBE activity in the host cell. Host cells for use in expressing the polypeptides encoded by the expression vectors of the present invention are well known in the art and include but are not limited to, bacterial cells, such as E. coli, or fungal cells, such as Saccharomyces cerevisiae or Pichia pastoris, insect cells, such as Drosophila S2 and Spodoptera Sf9, animal cells, such as CHO, COS, BHK, 293, and plant cells. Appropriate culture mediums and growth conditions for the above-described host cells are well known in the art. In at least one embodiment, the present disclosure provides a method for producing a cannabinoid comprising: (a) culturing in a suitable medium a recombinant host cell of the present disclosure; and (b) recovering the produced cannabinoid.

The BBE activity of the recombinant polypeptides of the present disclosure can result in the oxidative cyclization of a prenylated aromatic compound, such as CBGA, to form a cyclic compound, such as CBCA, is a critical enzymatic step in the biosynthesis of cannabinoids, and many other compounds of interest. Accordingly, it is contemplated that the recombinant polypeptides having BBE activity of the present disclosure can be incorporated in methods and compositions useful for a range of in vitro, cell-free systems, or in vivo, recombinant host cell systems for the biosynthesis of compounds requiring such an oxidative cyclization step.

In at least one embodiment, the present disclosure contemplates the use of the recombinant polypeptides with BBE activity in any in vitro, cell-free system that requires a biosynthetic oxidative cyclization step to produce a compound. Indeed, because they are expressed in soluble form in E. coli, the recombinant polypeptides of the present disclosure (e.g., polypeptides of Tables 3 and 4) can be incorporated directly into known cell-free biosynthesis systems and processes. For example, they can be used in methods and compositions for the cell-free biosynthesis of cannabinoid compounds, such as those methods and compositions described in e.g., Valliere et al. “A bio-inspired cell-free system for cannabinoid production from inexpensive inputs,” Nature Chemical Biology Vol. 16, December 2020, 1427-1433; and WO2020/028722A1, which are hereby incorporated by reference herein. Moreover, using the recombinant polypeptides with BBE activity of the present disclosure, the known cell-free cannabinoid biosynthesis methods can be carried out and result more efficient and selective conversion of CBGA, CBGVA, and/or CBGPA to the cyclized cannabinoid products, CBCA, CBCVA, and CBCPA, respectively.

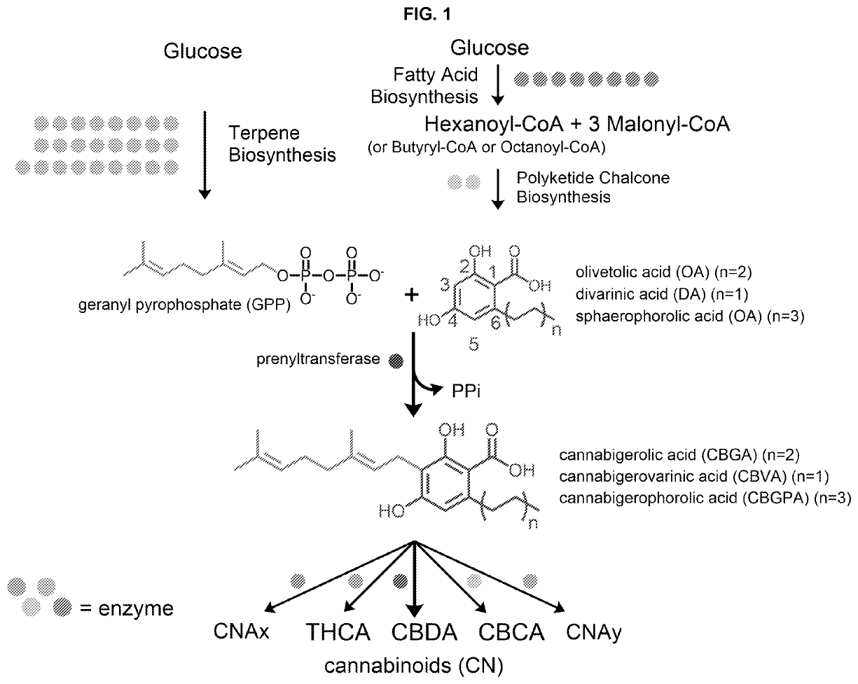

FIG. 1 depicts a schematic overview of the molecular inputs/outputs and enzymes involved in an exemplary system for the biosynthesis of cannabinoid compounds. In the right side of the scheme of FIG. 1, the input molecule glucose is converted via fatty acid biosynthesis enzymes to the precursor compounds, hexanoyl-CoA and malonyl-CoA. Or alternatively to hexanoyl-CoA, butyryl-CoA or octanoyl-CoA. The precursors, hexanoyl-CoA and malonyl-CoA are converted via polyketide chalcone biosynthesis enzymes to the cannabinoid precursor compounds, olivetolic acid (OA). Or alternatively, butyryl-CoA is converted to divarinic acid (DA), or the precursor octanoyl-CoA is converted to sphaerophorolic acid (PA). Each of the cannabinoid precursors OA, DA, or PA, is capable as acting as a cannabinoid precursor substrate compound, or as the “polyketide input,” for a prenyltransferase. The left side of this scheme of FIG. 1, depicts a terpene biosynthesis route that converts glucose input molecules to geranyl pyrophosphate (GPP), which is the co-substrate used by the prenyltransferase to convert the cannabinoid precursor compounds, OA, DA, or PA, to the corresponding cannabinoid product compounds, CBGA, CBGVA, or CBGPA. These exemplary cannabinoid product compounds differ only in the length alkyl carbon chain as shown by the generic structure depicted in FIG. 1.

As shown in the bottom of the scheme of FIG. 1, these cannabinoid products (e.g., CBGA, CBGVA, or CBGPA) are themselves precursor substrate compounds for a final step of oxidative cyclization. In C. sativa, this oxidative cyclization is carried out by the cannabinoid synthase enzymes (e.g., CBDAS, THCAS, or CBCAS) resulting in the cyclized cannabinoid compounds, THCA, CBDA, CBCA, and other structural analogs. These plant-derived synthase enzymes are associated with membranes and have proven difficult to incorporate into cell-free soluble biosynthesis systems. Accordingly, the present disclosure contemplates that the recombinant polypeptides having BBE activity that are expressed in soluble form in E. coli can be substituted for the various C. sativa synthase and carry out this final step of oxidative cyclization in such cell-free biosynthetic systems.

Accordingly, in at least one embodiment, the present disclosure provides compositions of enzyme useful in cell-free biosynthesis systems that comprise the recombinant polypeptide having BBE activity of the present disclosure (e.g., polypeptide of Tables 3 or 4), and one or more enzymes that are capable of producing a substrate that one desires to undergo an oxidative cyclization catalyzed by the recombinant polypeptide. In at least one embodiment, the composition can further comprise compounds that are substrates for the one or more enzymes that produce the substrate for the oxidative cyclization catalyzed by the recombinant polypeptide having BBE activity.

A wide range of the one or more enzymes can be incorporated in the composition depending on the ultimate biosynthetic product(s) desired from the system. In at least one embodiment, the composition comprises a prenyltransferase that converts a cannabinoid precursor compound (e.g., OA, DA, PA) and co-substrate, such as GPP, to a prenylated aromatic compound (e.g., CBGA, CBGVA, CBGPA). Thus, it is contemplated that a composition useful in the biosynthetic production of a cyclized cannabinoid (e.g., CBCA) can include a recombinant polypeptide of the present disclosure with BBE activity, a prenyltransferase (e.g., NphB), and substrates for the prenyltransferase (e.g., OA, DA, PA, and GPP).

One of skill will recognize that this simple cell-free biosynthetic composition could be further expanded on to include additional enzymes capable of producing the cannabinoid precursor compound and/or GPP substrate. Accordingly, in at least one embodiment of the composition, in addition to the recombinant polypeptide with BBE activity, the one or more enzymes of the biosynthetic system can include one or more of: (a) a plurality of enzymes that convert isoprenol or prenol to geranylpyrophosphate (GPP); (b) enzymes that convert a cannabinoid precursor (e.g., OA, DA, PA) to a prenylated aromatic compound (e.g., CBGA, CBGVA, CBGPA); (c) enzymes that convert malonate and acetyl-CoA to malonyl-CoA; and/or (d) enzymes that convert ADP and/or AMP to ATP; optionally, wherein the enzymes that convert ADP and/or AMP to ATP also convert acetyl-phosphate to acetic acid. Table 5 provides a list of exemplary enzymes that can be used in a cell-free biosynthesis system incorporating the recombinant prenyltransferase polypeptides of the present disclosure.

TABLE 5
Exemplary enzymes useful for cell-free biosynthesis

| Enzyme | | | |
|---|---|---|---|
| Abbreviation | Enzyme | Source Organism | Accession # |
| AAE3 | Acyl Activating Enzyme 3 | C. sativa | AFD33347.1 |
| MatB | Malonyl-CoA Synthetase | R. plaustris | CAE25665.1 |
| mdcA | Malonate Decarboxylase | Geobacillus sp. 44B | OQO99201.1 |
| | α subunit | | |
| PTA | Phosphotransacetylase | G. stearothermophilus | WP_053532564 |
| OLS | Olivetol Synthase | C. sativa | BAG14339.1 |
| OAC | Olivetolic Acid Cyclase | C. sativa | AFN42527.1 |
| NphB | Prenyltransferase | Streptomyces sp. | AWW43729.1 |
| ADK | Adenylate Kinase | G. thermodenitrificans | ABO65513 |
| Ppase | Pyrophosphatase | G. stearothermophilus | O05724 |
| CPK | Creatine Kinase | Rabbit Muscle | Sigma Aldrich |
| ThiM | Hydroxyethylthiazole kinase | E. coli | NP_416607 |
| IPK | Isopentenyl Kinase | M. jannaschii | WP_01069535 |
| IDI | Isopentyl diphosphate | E. coli | NP_417365 |
| | isomerase | | |
| FPPS S82F | Farnesyl Pyrophosphate | G. stearothermophilus | KOR95521 |
| | Synthase | | |

As described herein, the recombinant polypeptides with BBE activity and altered product selectivity and/or increased activity of the present disclosure can be incorporated in any biosynthesis method requiring a BBE catalyzed biocatalytic step. Thus, in at least one embodiment, the recombinant polypeptides (e.g., exemplary polypeptides of Tables 3 and 4) can be used in a method for preparing a cannabinoid compound of structural formula (I)

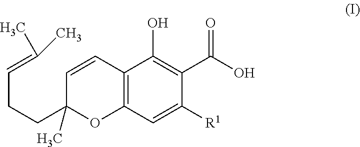

wherein, R¹ is C1-C7 alkyl. This biosynthetic method comprises contacting an engineered polypeptide of the present disclosure (e.g., polypeptide of any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82) under suitable reactions conditions, with a compound of structural formula (II)

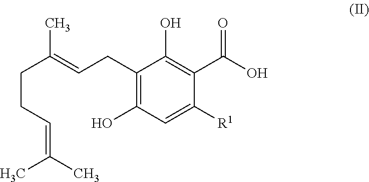

wherein, R¹ is C1-C7 alkyl.

Exemplary cyclized cannabinoid compounds of structural formula (I) (CBCA, CBCVA, CBCPA) that can be prepared in stereoselective excess using the recombinant polypeptides of the present disclosure are depicted in FIG. 2. FIG. 3 depicts a proposed mechanism of the biocatalytic reaction carried out by a recombinant polypeptide of the present disclosure (e.g., polypeptide of Tables 3 or 4) to provide the cyclized cannabinoid products, CBCA, CBCVA, CBCPA. The cannabinoid compound substrate, cannabigerolic acid (CBGA) can be converted in stereoselective excess to the cyclized cannabinoid compound product, CBCA rather than the alternative cyclized product, THCA. Similarly, the cannabinoid compound substrate, cannabigerolic acid (CBGVA) can be converted in stereoselective excess to the cyclized cannabinoid compound product, CBCVA rather than the alternative cyclized product, THCVA. Accordingly, it is contemplated that the recombinant polypeptides of the present disclosure will exhibit BBE activity with other cannabinoid compounds that are structural analogs of the prenylated aromatic cannabinoids, CBGA, CBGVA, and CBGPA, including but not limited to the exemplary cannabinoid compounds listed in Table 1.

Accordingly, in at least one embodiment of the method, the compound of structural formula (I) is cannabichromenic acid (CBCA) and the compound of structural formula (II) is cannabigerolic acid (CBGA); the compound of structural formula (I) is cannabichromevarinic acid (CBCVA) and the compound of structural formula (II) is cannabigerovarinic acid (CBGVA); or, the compound of structural formula (I) is cannabichromephorolic acid (CBCPA) and the compound of structural formula (II) is cannabigerophorolic acid (CBGPA)

The present disclosure contemplates ranges of suitable reaction conditions that can be used in the methods, including but not limited to ranges of pH, temperature, buffer, solvent system, substrate loading, polypeptide loading, co-substrate or co-factor loading, atmosphere, and reaction time. The present disclosure also contemplates that the methods comprising the biocatalytic conversion of a substrate compound of structural formula (II) to a product compound of structural formula (I) using an recombinant polypeptide with BBE activity of the present disclosure can further comprise additional chemical or biocatalytic steps carried out on the product compound, product compound work-up, extraction, isolation, purification, and/or crystallization, each of which can be carried out under a range of conditions.

Further suitable reaction conditions for carrying out the biocatalytic conversion of a substrate compound of structural formula (II) to a product compound of structural formula (I) using a recombinant polypeptide with BBE activity described herein can be readily optimized by routine experimentation that includes, but is not limited to, contacting the recombinant polypeptide and substrate under experimental reaction conditions of concentration, pH, temperature, solvent conditions, and detecting the production of the desired compound of structural formula (I), for example, using the methods described in the Examples provided herein.

Generally, a biosynthetic reaction involving a recombinant polypeptide catalyzed conversion of a cannabinoid compound of formula (II) to a cannabinoid product of formula (I) can be carried out in accordance with reaction conditions for cell-free biosynthesis of cannabinoids known in the art (see e.g., Valliere et al. 2020; or WO2020028722A1) or as described herein. In some embodiments of the method, the suitable reaction conditions can include a temperature range of about 293 K to about 318 K. In one embodiment, the suitable reaction conditions comprise a temperature of about 310 K.

It is also contemplated that the increased thermostability of the recombinant polypeptides with BBE activity of the present disclosure can allow a range of substrate loading in the reaction. Thus, in some embodiments of the method of preparing a cannabinoid compound of structural formula (I), the suitable reaction conditions can comprise a cannabinoid substrate loading of at least about 0.6 g/L, at least about 1.2 g/L, 2 g/L, 6 g/L, 12 g/L, 18 g/L, 24 g/L, 30 g/L or even greater. Specifically, where the cannabinoid substrate is selected from CBGA, CBGVA, and CBGPA, the substrate loading can be at least about 0.6 g/L, at least about 1.2 g/L, 2 g/L, 6 g/L, 12 g/L, 18 g/L, 24 g/L, 30 g/L, or even greater

The recombinant polypeptides with BBE activity of the present disclosure can allow reactions to be carried out with higher rates of biocatalytic conversion. Thus, it is contemplated that in some embodiments the recombinant polypeptide catalyzed conversion of a cannabinoid compound of formula (II) to a cannabinoid product of formula (I) can be carried out with lower concentrations of the recombinant polypeptide with BBE activity. Accordingly, in at least one embodiment of the method, the suitable reaction conditions comprise a recombinant polypeptide concentration of about 0.1 g/L to about 5 g/L, or even lower concentration.

As noted elsewhere herein, suitable pH and buffer conditions for the biosynthesis of cannabinoids are known in the art and can also be used with the recombinant polypeptides with BBE activity of the present disclosure. Accordingly, in at least one embodiment a method of producing a cannabinoid compound of structural formula (I) using the engineered polypeptides of the present disclosure, the suitable reaction conditions can comprise: (a) a pH of about 5.0 to about 11.0, or about 4.0 to 10.0; and/or a buffer solution of about 0.05 M Tris-CI pH 8.0 to about 0.5 M Tris-Cl pH 8.0. In at least one embodiment, the suitable reaction conditions for preparing the cannabinoid compound, CBCA, comprise: cannabigerolic acid (CBGA), 0.1 M buffer pH 8.0, and the recombinant polypeptide at 310 K for at least 1 hour. It is contemplated that identical or very similar conditions for the biosynthetic production of CBCVA or CBCPA. Suitable reaction conditions for the various recombinant polypeptides of the present disclosure can be easily determined using routine techniques for optimizing biocatalytic reaction conditions well-known to one of ordinary skill.

In at least one embodiment, the recombinant polypeptides with BBE activity of the present disclosure can be further engineered for use in a biosynthetic reaction for the production of a cyclized cannabinoid compound, or a composition comprising a cyclized cannabinoid compound. It is contemplated that the produced cannabinoid compound can include, but is not limited to, the cannabinoid compounds of Table 1. Accordingly, in at least embodiment, the biosynthetic reaction can be used for production of a cyclized cannabinoid compound selected from cannabichromenic acid (CBCA), cannabichromene (CBC), cannabichromenic acid isomer B (CBCA-B), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

A cell-free biosynthetic reaction using the recombinant polypeptides of the present disclosure can be carried out using a range of biocatalytic reaction methods. For example, the pathway enzymes can be purchased commercially, mixed in a suitable buffer with the recombinant polypeptides with BBE activity of the present disclosure, and then the solution is exposed to the suitable substrate, and incubated under conditions suitable for production of the desired cannabinoid compound. In some embodiments, it is contemplated that one or more of the pathway enzymes can be bound to a solid support. It is also contemplated that one or more of the pathway enzymes can be expressed using phage display or other surface expression system and, for example, fixed in a fluid pathway corresponding to points in the metabolic pathway's cycle.

It is also contemplated that one or more polynucleotides encoding the one or more pathway enzymes can be cloned into one or more host cells under conditions providing expression of the enzymes. The host cells can then be lysed and the lysate comprising the one or more enzymes (including the recombinant polypeptides with BBE activity) can be combined with a suitable buffer and substrate (and one or more additional enzymes of the pathway, if necessary) to produce the desired cannabinoid. Alternatively, the enzymes can be isolated from the lysed preparations with or without heat treatment and then recombined in an appropriate buffer.

In at least one embodiment of a method for producing a cannabinoid, a heterologous nucleic acid encoding a recombinant polypeptide having BBE activity and altered product selectivity and/or increased activity, (e.g., an exemplary recombinant polypeptide of Tables 3 or 4) can be introduced into a recombinant host cell. The recombinant host cell can then be used for production of the polypeptide or incorporated in a biocatalytic process that utilizes the BBE activity of the recombinant polypeptide expressed by the host cell for the catalytic oxidative cyclization of a substrate, e.g., the cyclization of CBGA to produce CBCA. In at least one embodiment, the recombinant host cell can further comprise a pathway of enzymes capable of producing a cannabinoid substrate (e.g., CBGA) in addition to the recombinant polypeptide with BBE activity of the present disclosure. It is contemplated that a recombinant host cell comprising a heterologous nucleic acid encoding a recombinant polypeptide of the present disclosure can provide improved biosynthesis of a cyclized cannabinoid (e.g., CBCA) in terms of titer, yield, and production rate, due to the improved conversion rate and/or product selectivity of the expressed BBE activity.

Accordingly, in at least one embodiment, the present disclosure provides a method of producing a cannabinoid derivative. wherein the method comprises (a) culturing in a suitable medium a recombinant host cell of the present disclosure; and (b) recovering the produced cannabinoid derivative. In at least one embodiment, the method of producing a cannabinoid derivative further contacting a cell-free extract of the culture containing the produced cannabinoid with a biocatalytic reagent or chemical reagent capable of converting the cannabinoid to a cannabinoid derivative. In at least one embodiment, the biocatalytic reagent is an enzyme capable of converting the produced cannabinoid to a different cannabinoid or a cannabinoid derivative compound. In at least one embodiment, the chemical reagent is capable of chemically modifying the produced cannabinoid to produce a different cannabinoid or a cannabinoid derivative compound. In at least one embodiment of the method for producing a cannabinoid, the method can further comprise contacting a cell-free extract of the culture containing the produced cannabinoid with a biocatalytic reagent or chemical reagent.

It is contemplated that the cannabinoid, or cannabinoid derivative produced using the methods of the present disclosure can be produced and/or recovered from the reaction in the form of a salt. In at least one embodiment, the recovered salt of the cannabinoid, or cannabinoid derivative is a pharmaceutically acceptable salt. Such pharmaceutically acceptable salts retain the biological effectiveness and properties of the free base compound.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1

### Preparation and Characterization of Clz9 Wild-Type BBE and Engineered Clz9 Variants

This example illustrates the preparation of a recombinant polypeptide with BBE activity, wild-type Clz9 of SEQ ID NO: 2, as well as a range of site-directed mutants of this parent wild type polypeptide. The example further illustrates screening of the site directed mutant for activity in converting the cannabinoid substrate, CBGA to the cyclized cannabinoid product, CBCA relative to the recombinant wild-type polypeptide of SEQ ID NO: 2.

### Materials and Methods

A. Cloning of Wild-Type Clz9 into pET28a with or without MBP

The codon-optimized gene of SEQ ID NO: 1 which encodes the Clz9 from Streptomyces sp. CNH287 was synthesized and cloned into the pET28a expression vector as follows: The amino acid sequence for the Clz9 BBE from Streptomyces sp. CNH287 was provided to Twist DNA. The sequence was codon optimized by Twist DNA along with addition of 20 bp complementary to the 20 bp upstream of the Ndel site and 20 bp downstream of the Xhol site for cloning via Gibson into the pET28a expression vector. The resulting cloned Clz9 construct has a 6×-HIS tag for expression in Escherichia coli. For cloning of Clz9 fused to MBP, a pET28a vector with MBP cloned between Ndel and Xhol was used as the recipient vector and Clz9 was cloned downstream of MBP as described above for the pET28a construct.

### B. Expression of MBP-Clz9

Expression of the recombinant Clz9 in E. coli having amino acid sequence of SEQ ID NO: 2 was carried out as follows: The clonal gene in the pET28a expression vector or pET28a-MBP expression vector was transformed into BL21-Gold(DE3) competent cells using standard chemical transformation methods. A single colony was used to inoculate 4 mL LB+kanamycin (50 mg/mL), which was grown at 37° C. and 250 rpm. After 12 hours, the overnight was used to inoculate 1 L LB+kanamycin (50 mg/mL). At an OD₆₀₀ of ˜0.6, the culture was induced with the addition of 0.4 mM isopropyl β-d-1-thiogalactopyranoside (IPTG) and grown at 18° C. and 250 rpm. After 12 hours, protein purification was carried out using standard Ni-NTA methods.

### C. Site-Directed Mutagenesis of Clz9

Mutant genes encoding the Clz9 variants were obtained through site directed mutagenesis using a mutagenic primer containing the desired mutation. The wild-type Clz9 gene of SEQ ID NO: 1 was used as the template to introduce mutations via polymerase chain reaction (PCR) using the mutagenic primer. Mutations were confirmed by Sanger sequencing. Following confirmation, expression of the recombinant Clz9 variants was carried out in E. coli.

The clonal gene in the pET28a expression vector was transformed into BL21-Gold (DE3) competent cells using standard chemical transformation methods. A single colony was used to inoculate 4 mL LB+kanamycin (50 mg/mL), which was grown at 37° C. and 250 rpm. After 12 hours, the overnight was used to inoculate 1 L LB+kanamycin (50 mg/mL). At an OD₆₀₀ of ˜0.6, the culture was induced with the addition of 0.4 mM isopropyl β-d-1-thiogalactopyranoside (IPTG) and grown at 18° C. and 250 rpm. After 12 hours, protein purification was carried out using standard Ni-NTA methods.

### D. BBE Activity Assay

To assay for BBE activity, 98 μL of the Clz9 variant polypeptide samples at 2 mg/ml concentration in 25 mM Tris pH 7.5 were mixed with 2 μL of 5 mM CBGA. The BBE reactions (100 μL final) were allowed to proceed for 4 hours at 32° C. and then quenched at by adding 900 μL methanol. Protein precipitate was removed by centrifugation (3 min at 16,000 g), and CBGA, THCA, and CBCA analyzed by HPLC. Relative Activity corresponds to the activity of the wild-type or variant Clz9 in the cell-free conversion rate of CBGA to CBCA in 4 hours at 32° C. under the assay conditions described above. Under these conditions wild-type Clz9 converts ˜50% of the input CBGA to CBCA. Relative rates are indicated as follows: +=<25% conversion at 4 hours; ++=>50% conversion at 4 hours; +++=>75% conversion at 4 hours; ++++=100% conversion at 4 hours; −=no conversion.

### Results

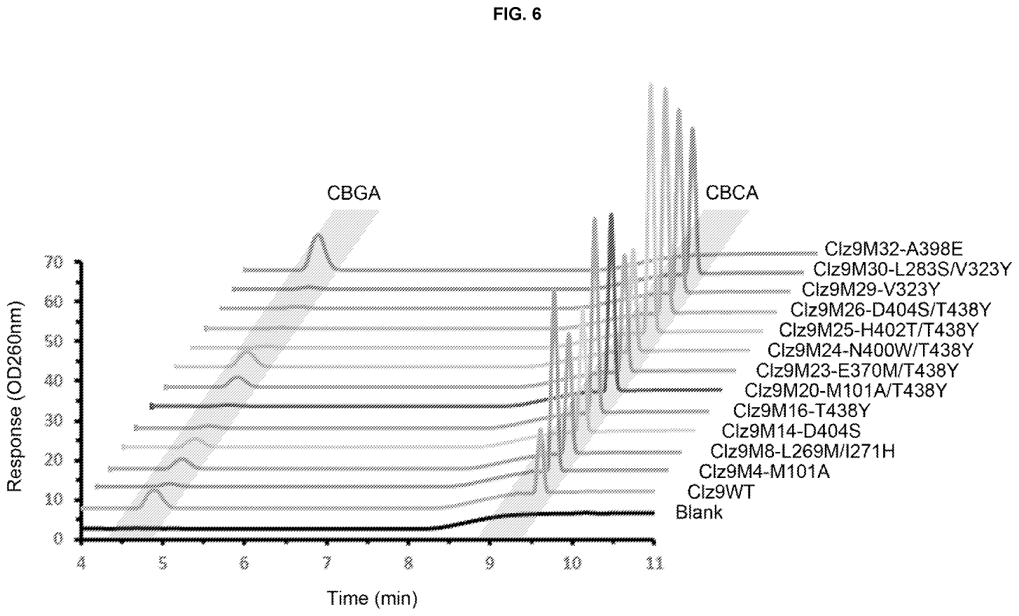

Table 4 provides a summary of the specific site-directed mutations found in 34 recombinant variants of the Clz9 polypeptide of SEQ ID NO: 2 that were prepared and screened for activity. FIG. 5 depicts an alignment of four of the variants (Clz9M1, Clz9M2, Clz9M3, and Clz9Mut34) with the wild-type Clz9 sequence of SEQ ID NO: 2. FIG. 6 shows raw HPLC data from the screening assays showing the relative production of CBCA (and depletion of substrate, CBGA) by 12 of the Clz9 variants. Table 4 summarizes the relative activity of the 34 Clz9 variants compared to the activity of the parent wild-type of SEQ ID NO: 2 under the same assay conditions.

### Example 2

### Identification of Bacterial Homologs of Clz9 and Preparation of Additional Recombinant Polypeptides with BBE Activity

This example illustrates the preparation, and characterization of a series of recombinant polypeptides with BBE activity from bacterial sources that were identified based on homology to wild-type Clz9 of SEQ ID NO: 2.

### Materials and Methods

### A. Homology Searching Using Wild-Type Clz9

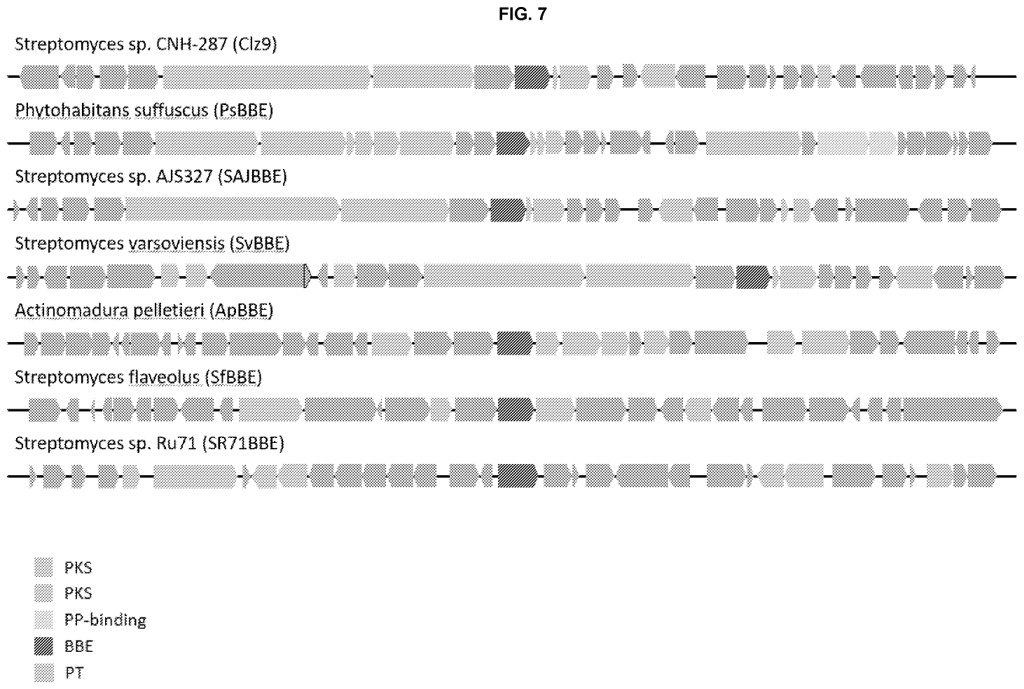

The sequence of the wild-type Clz9 of SEQ ID NO: 2, which exhibits BBE activity in the conversion of CBGA to CBCA, was used to search for bacterial homologs that clustered with either a putative PKS gene cluster and/or a prenyltransferase (NphB homolog). In order to identify clusters, the nucleotide sequence for NphB or Clz9 (SEQ ID NO: 1) was used as template for a BlastN search. Resulting positive hits with between 25%-90% identity were analyzed further by submitting the genome region 40 kb upstream and 40 kb downstream (80 kb total) to 2ndfind (See at: biosyn.nih.go.jp/2ndfind/). The resulting annotated genes and clusters were then analyzed for the presence of a BBE in addition to a prenyltransferase, polyketide synthase or both. Representative BBE gene clusters from the various bacterial source organisms that contain polyketide synthase (PKSs) and/or prenyltransferase enzymes are depicted in FIG. 7. Putative bacterial homologs of Clz9 with BBE activity were identified in Streptomyces flaveolus (SflBBE), Streptomyces sp. Ru71 (SR71BBE), Phytohabitans suffuscus (PsBBE), Streptomyces sp. AJS327 (SAJBBE), Streptomyces varsoviensis (SvBBE), and Actinomadura pelletieri (ApBBE). A summary of these bacterial homologs is provided in Table 3 and the accompanying Sequence Listing.

### B. Cloning and Expression of Bacterial Homologs of Clz9

Genes encoding the identified bacterial homolog polypeptides of SEQ ID NO: 4, 6, 8, 10, 12, and 14, were constructed, cloned and expressed in E. coli fused to MBP as described above for Clz9. Expression for bacterial homologs or Clz9 transformed in E. coli BL21Gold (DE3) were induced at an OD₆₀₀ of ˜0.6 with the addition of 0.4 mM isopropyl β-d-1-thiogalactopyranoside (IPTG) and grown at 18° C. and 250 rpm for an additional 16 hours. After 16 hours, protein purification was carried out using standard Ni-NTA methods.

### D. BBE Activity Assay

The bacterial homolog polypeptides of SEQ ID NO: 4, 6, 8, 10, 12, and 14, were assayed for BBE activity in the conversion of CBGA to CBCA as described in Example 1.

### Results

Table 3 provides a summary of the recombinant polypeptides of the bacterial homologs of Clz9 screened and their BBE activity relative to Clz9.

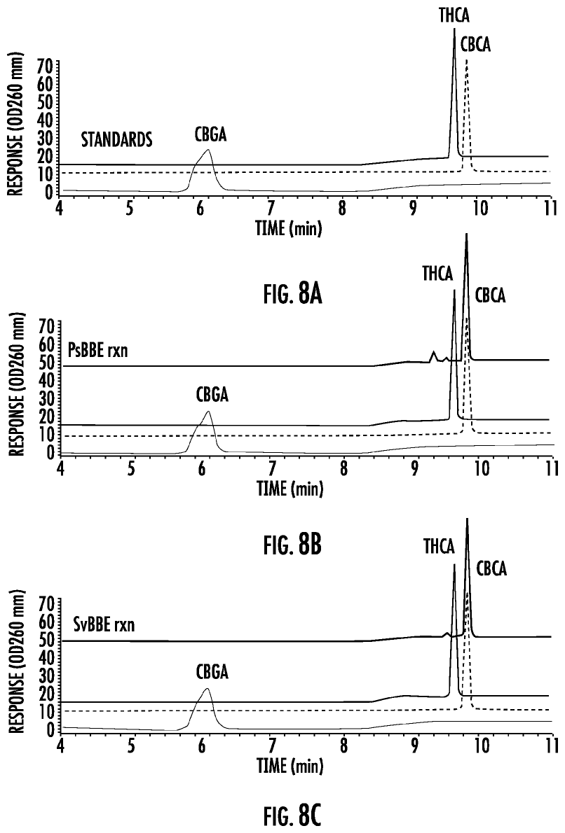

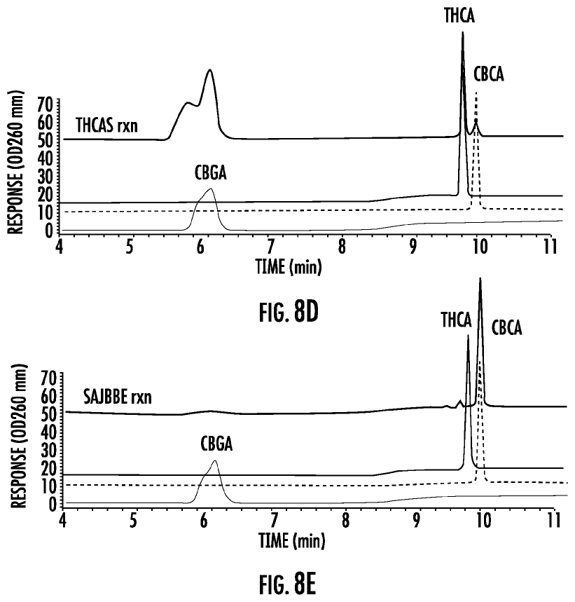

FIGS. 8B, 8C, and 8E depict HPLC results showing products of cyclization reactions catalyzed at pH 7.5 by the BBE homologs from Phytohabitans suffuscus (PsBBE) (FIG. 8B), Streptomyces varsoviensis (SvBBE) (FIG. 8C), and Streptomyces sp. AJS327 (SAJBBE) (FIG. 8E), carried out as described in Example 2. These active bacterial BBE homologs of Clz9 produce CBCA exclusively at all pH values tested. In contrast, THCAS from C. sativa (FIG. 8D) produces primarily CBCA with some THCA at pH 7.5 and produces primarily THCA with some CBCA at pH 5.5. The profiles of the standards for the cannabinoid substrate CBGA, and the cannabinoid products THCA and CBCA, are shown in FIG. 8A.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A method for preparing a compound of structural formula (I) 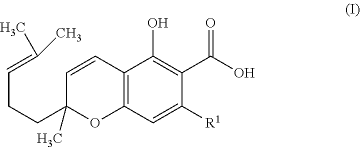
wherein, R¹ is C1-C7 alkyl,
comprising contacting under suitable reactions conditions a compound of structural formula (II) 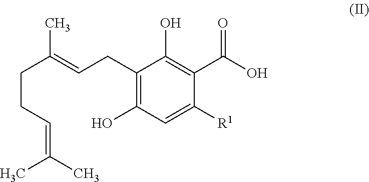
wherein, R¹ is C1-C7 alkyl,
and a recombinant polypeptide comprising an amino acid sequence of at least 80%, at least 90%, at least 95%, at least 97%, at least 98%, or at least 99% identity to a sequence selected from SEQ ID NO: 8, 2, 4, 6, 10, 12, and 14.

2. The method of claim 1, wherein the suitable reaction conditions comprise:
(a) a cell-free solution;
(b) a substrate compound of structural formula (II), 0.1 M buffer pH 8.0, and the recombinant polypeptide at 298 K for at least 1 hour;
(c) a loading of a substrate compound of structural formula (II) of at least about 0.6 g/L, at least about 1.2 g/L, 2 g/L, 6 g/L, 12 g/L, 18 g/L, 24 g/L, 30 g/L or even greater; and/or (d) a recombinant polypeptide concentration of about 0.1 g/L to about 5 g/L, or even lower concentration.

3. The method of any one of claims 1-2, wherein:
(a) the compound of structural formula (I) is cannabichromenic acid (CBCA) and the compound of structural formula (II) is cannabigerolic acid (CBGA);
(b) the compound of structural formula (I) is cannabichromevarinic acid (CBCVA) and the compound of structural formula (II) is cannabigerovarinic acid (CBGVA); or
(c) the compound of structural formula (I) is cannabichromephorolic acid (CBCPA) and the compound of structural formula (II) is cannabigerophorolic acid (CBGPA).

4. A composition comprising: (a) a recombinant polypeptide having BBE activity comprising an amino acid sequence of at least 80%, at least 90%, at least 95%, at least 97%, at least 98%, or at least 99% identity to a sequence selected from SEQ ID NO: 8, 2, 4, 6, 10, 12, and 14; and (b) one or more enzymes that produce a substrate for the recombinant polypeptide.

5. The composition of claim 4, wherein the one or more enzymes:
(a) comprise enzymes that produce a prenylated aromatic compound; optionally, wherein the prenylated aromatic compound is selected from: CBGA, CBGVA, CBGPA, and a combination thereof;
(b) comprise an enzyme that is a Prenyltransferase; optionally, wherein the Prenyltransferase is NphB;
(c) comprise enzymes that convert isoprenol or prenol to geranylpyrophosphate (GPP);
(d) comprise enzymes that convert malonate and acetyl-CoA to malonyl-CoA;
(e) comprise enzymes that convert ADP and/or AMP to ATP; optionally, wherein the enzymes that convert ADP and/or AMP to ATP also convert acetyl-phosphate to acetic acid;
(f) comprise Acyl activating enzyme 3 (AAE3); Olivetol synthase (OLS); Olivetolic acid cyclase (OAC); and Prenyltransferase (NphB); and/or
(g) comprise Acetyl-phosphate transferase (PTA); Malonate decarboxylase alpha subunit (mdcA); Acyl activating enzyme 3 (AAE3); Olivetol synthase (OLS);
Olivetolic acid cyclase (OAC); Prenyltransferase (NphB); Hydroxyethylthiazole kinase (ThiM); Isopentenyl kinase (IPK); Isopentyl diphosphate isomerase (IDI);
Diphosphomevalonate decarboxylase alpha subunit (MDCa); and Geranyl-PP synthase (GPPS) or Farnesyl-PP synthase mutant S82F (FPPS S82F).

6. The composition of any one of claims 4-5, wherein the composition further comprises geranylpyrophosphate (GPP) and a cannabinoid precursor substrate selected from olivetolic acid (OA), divarinic acid (DA), and sphaerophorolic acid (PA).

7. The composition of any one of claims 4-5 in which the composition is a cell-free solution.

8. A recombinant polypeptide having berberine bridge enzyme (BBE) activity comprising an amino acid sequence of at least 80% identity to SEQ ID NO: 2 and amino acid residue differences as compared to SEQ ID NO: 2 at one or more positions selected from T325, F156, L238, L283, T325, and G340; optionally, wherein the amino acid residue differences are selected from: T325G, F156Y, L238S, L283S, and G3401.

9. The polypeptide of claim 8, wherein the polypeptide further comprises amino acid residue differences as compared to SEQ ID NO: 2 at one or more positions selected from M101, A171, N267, L269, 1271, V323, E370, A398, N400, H402, D404, and/or T438; optionally, wherein the amino acid residue differences are selected from: M101A, A171Y, N267V, L269M, 1271H, V323Y, E370M, A398E, N400W, H402T, D404S, and/or T438Y

10. The polypeptide of claim 9 in which the polypeptide comprises a set of at least two amino residue differences selected from: M101A/T438Y; M101A/L269M/1271H; A171Y/A398E; L269M/1271H; L269M/1271H/T438Y; 1271H/T438Y; L283S/V323Y; E370M/T438Y; N400W/T438Y; N400W/H402T/D404S; N400W/H402T/D404S/T438Y; H402T/T438Y; and/or D404S/T438Y.

11. The polypeptide of any one of claims 8-10 in which the polypeptide comprises an amino acid sequence of at least 80%, at least 90%, at least 95%, at least 97%, at least 98%, or at least 99% identity to a sequence selected from the group consisting of SEQ ID NO: 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

12. The polypeptide of any one of claims 8-11 in which the BBE activity of the polypeptide as compared to a polypeptide consisting of SEQ ID NO: 2 is increased at least 1.2-fold, at least 1.5-fold, at least 2-fold, at least 5-fold, or more.

13. The polypeptide of claim 12 in which the BBE activity is measured as the rate of conversion of the substrate cannabigerolic acid (CBGA) to the products, THCA and/or CBCA, under reaction conditions of 0.05 mM CBGA, 50 mM Tris at pH 5.5-9.5 and 298 K.

14. A polynucleotide encoding the polypeptide of any one of claims 8-13.

15. The polynucleotide of claim 14 in which the polynucleotide sequence comprises a sequence of at least 80%, at least 90%, at least 95%, at least 97%, at least 98%, or at least 99% identity to a sequence selected from the group consisting of SEQ ID NO: 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, and 81.

16. An expression vector comprising the polynucleotide of any one of claims 14-15.

17. The expression vector of claim 16 comprising a control sequence.

18. A host cell comprising the polynucleotide of any one of claims 14-15 or the expression vector of any one of claims 16-17.

19. A method for preparing a polypeptide of any one of claims 8-13 comprising culturing a host cell of claim 18 and isolating the polypeptide from the cell.


SEQUENCE LISTING (82 sequences)

SEQ ID NO: 1 - DNA, 1521 bp, Streptomyces sp.
  source 1..1521: mol_type = genomic DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 2 - protein, 506 aa, Streptomyces sp.
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 3 - DNA, 1518 bp, Streptomyces flaveolus
  source 1..1518: mol_type = genomic DNA
atgattaatcgccgtagttttctgacgggagcgggtggggtagccctcgctactacggcgctcacgggagcagcccaggccaaaggtgatgcgttcggtacccgttggtctcgccttgccgaccgcttacagggacgtctcgtcttaccgagtgacgcggattatcagcgtgccaaacagctgtaccaagttcagttcgacgcaactagcccgcgtgcagtggcgtattgtgtcagcgcagcagacgtaagcgcctgtgttcgcttcgcagaggcctacggtattccagtagcggcacgctctggcggtcactcagcaggtggctacagcaccactgaaggcctcgttatcgacgtaagtggcttaaacagtattgttcttggtgagtcatccgtccgtatgggccccggtgcacagttagtagacattggcgatacccttgcacccgcagggttgggcatttctggtggctactgccccaccgtggccgcgggtggcttcttccaaggtggtgggatggggttgttcacgcgctcggtcggtattgcatccgataaagttaacagtgcgcaagtagtgctggctgacggccgcgttgtcaccgcgtcccctcgcagtcactctgacttgttctgggctctgcgtggcggcggcggtggtaatttcggcatcgtcacttcctacgacgtgacgccttctccgctgacggacgttgccgccatcaacctggtttggactttcgaccaaagtcttgacatgctcgatggatggactcgttggctgcccgatgcgccgtggaccatcggtagcggggtgaacgtcaccttgacagacgcagggccaggcaccgtgccgacagcttcagttttcttagggagtgtggacactggagccggcttcgaggcggaaatcgcccgtctgatttcgcttgtgggacgcccacctgtatatcaacaaaagtttacagctccatatcgcagcgtactgatgtcgttataccaatgttcagacctgacggcccaacaatgtcaccgtgccgacacatccccagacggtcgtattcaacgcccagccttcggtgcatggcgctcccgcttattcgaacgcactatgccacgcgaggggtgggcaaaggctttggaaacgttcaacgcgaatcgcttcgccggccagatgcgtcagcttcaaatctctgctctgggtggtcgcgccaatacggtggctcgcgacgcgactgcgtacgtgcatcgtgactcgttgttttcagtctcataccttactagcaatgcggccgctccagttgctgacgaggccaaggcagcggcatgggagtacgtagacgacggatttgcagtaatcgacccatatagtaacggagaaacatatcaaaatttcatcgaccctcgtctgccggattggcgccgctcttactacgcagagaactatgcgcgtttagcgcgcgtcaaggataagtatgacccatatcgtttcttccgtttcgcgcaaggtgtgcgttaa

SEQ ID NO: 4 - protein, 505 aa, Streptomyces flaveolus
MINRRSFLTGAGGVALATTALTGAAQAKGDAFGTRWSRLADRLQGRLVLPSDADYQRAKQLYQVQFDATSPRAVAYCVSAADVSACVRFAEAYGIPVAARSGGHSAGGYSTTEGLVIDVSGLNSIVLGESSVRMGPGAQLVDIGDTLAPAGLGISGGYCPTVAAGGFFQGGGMGLFTRSVGIASDKVNSAQVVLADGRVVTASPRSHSDLFWALRGGGGGNFGIVTSYDVTPSPLTDVAAINLVWTFDQSLDMLDGWTRWLPDAPWTIGSGVNVTLTDAGPGTVPTASVFLGSVDTGAGFEAEIARLISLVGRPPVYQQKFTAPYRSVLMSLYQCSDLTAQQCHRADTSPDGRIQRPAFGAWRSRLFERTMPREGWAKALETFNANRFAGQMRQLQISALGGRANTVARDATAYVHRDSLFSVSYLTSNAAAPVADEAKAAAWEYVDDGFAVIDPYSNGETYQNFIDPRLPDWRRSYYAENYARLARVKDKYDPYRFFRFAQGVR

SEQ ID NO: 5 - DNA, 1518 bp, Streptomyces sp.
  source 1..1518: mol_type = genomic DNA
atggtaaatcgtcgtagcttcttggtgggcgctggtggcgctgccttggctgccacgggcttagcaagcccagcctttgcacgtagttcccgcggcggtaaatggcaccgtttatcgcaccatcttggtgggcgcttggtcttaccatctgatgccgactatccgacggccaagcaactgtacctcgctcaatttgatcacattcacccgcaagcgatcgcctactgtactagcccggcggacgtagccctgtgcttgcgctttgcggagtaccacgatgttcccattgcagtgcgctctggtgggaattgcgcaggcggttatagcaccaagcctgactctttggtcattgacgttagtcaaatgaatcaaatttcgatcggctctaaatcggttaagttaggtcctggagcagccctcgtcgacatcaccgacaaattaagtcgcaacggtctgagtattgcagacggttattgcccctcggtcgcggcaggcggattcctgtcaggcggcggtgccggaccatttacccgtgcaatgggcatggcaaccgatagtgtaaccaaggcccaggtggtcttggcggatggtcgcttagtaacagcgagcccgcacgagaatccagacttatactgggcggtgcgcggggggggcggtggcaatttcggtgttgtgactggctaccaaattacaccggaacccctcaccgacttggacacgtgtactttattctggccatgggaccagggcttagaggtgttcgaaggctggtcaaagtggcaagctgacgctccatgggccatcggatccgcattgctcatcactttgatggatgccgcacctggtaaagtaccagtgctgagtgttttcatgggctcagtggatacgggcggcattttagcgccagaagttgagcgcctgatttcactggttgggcgcccacccgcacgtcgccaacctgtaaaggctccgtacaagaagatcatgatggggttttataattgtgacgacaaaacggtggacgagtgtcaccgcgtgtcgacttggccggacgcgttactcccgcgcatggagtatggtatctggcgttcgcgtttgttttctaaaccactgccccgcggcggctggagcgatgttctgcaagtctttgaggatgaacgttggccgggacagatgcgccagcttcaactcgtcaccatgggtggtaaggccaacaccgtcgcccgtgatgccacggcgtttgtccatcgcgacacaattttctccatcgcctacttagcggcgaattataccgggccggtaagtgccgagaccgttacatcagcagaacatttcgcggacaccggcttcaacgtaattgatccgtatagcaatggggagacatatttaaacttctatgatccacgcgatcccaactgggagaagagttattacgcagaaaattatccgcgtcttatgcgtatcaaggacaaatacgatccatacaacatctttaaccacgaccaatctgtgcgctaa

SEQ ID NO: 6 - protein, 505 aa, Streptomyces sp.
MVNRRSFLVGAGGAALAATGLASPAFARSSRGGKWHRLSHHLGGRLVLPSDADYPTAKQLYLAQFDHIHPQAIAYCTSPADVALCLRFAEYHDVPIAVRSGGNCAGGYSTKPDSLVIDVSQMNQISIGSKSVKLGPGAALVDITDKLSRNGLSIADGYCPSVAAGGFLSGGGAGPFTRAMGMATDSVTKAQVVLADGRLVTASPHENPDLYWAVRGGGGGNFGVVTGYQITPEPLTDLDTCTLFWPWDQGLEVFEGWSKWQADAPWAIGSALLITLMDAAPGKVPVLSVFMGSVDTGGILAPEVERLISLVGRPPARRQPVKAPYKKIMMGFYNCDDKTVDECHRVSTWPDALLPRMEYGIWRSRLFSKPLPRGGWSDVLQVFEDERWPGQMRQLQLVTMGGKANTVARDATAFVHRDTIFSIAYLAANYTGPVSAETVTSAEHFADTGFNVIDPYSNGETYLNFYDPRDPNWEKSYYAENYPRLMRIKDKYDPYNIFNHDQSVR

SEQ ID NO: 7 - DNA, 1422 bp, Phytohabitans suffuscus
  source 1..1422: mol_type = genomic DNA
atgtcaacggcccctgtgtttggtggacgcatcgtcacgcgtctggatccgtcttatgaggaagctcgccaggtgtggaatgcagtcccggaccaccgtccattggagatccgttactgccgcgacgctcaagatgttgccgcggcacttcgtgatgcgcttgcccgcggattacctttcgccgcacgcagtggtggtcacagtgctgccgcactgtccgtcgtgccagatggagtagttattgacgtaagtcccttaaacagcatcagcctggaccaggacgcatcacaagttacatttggctccggtgccactttaggggcaatctatcgcacactctgggagtcaggcgtcacggttccggccggcacgtgcccaaatattggtgtggcgggtcacgttctcggcggcggtttggggattcttagtcgttcacgtggcccgctggtggaccatctcattggagtcactatggtggacgcccgtggtgaattagtgaccgcagacgaagaaaatcactccgacttgctgtgggcctgccgcggcggcggcggtgggaattacggcatcgcgacgagtttcacgttgcgcactcagcctatcgcggatgtcacactgttcaatattacatggcgcttcgatgacctcggacccgccgtgaaggcctggcagtcatggctcgccgatgccgaccatcgtatcaatccctttatgatgctttacccccgcgagcaagacatggtagcgaccgttggagtcatggaagggacgccagacgctttcgaaccgctggttaagcctctgcttgacgcggtggagccaagtgaggtggtgattgaaccgatgtcgttcatccaagccgtggatactgtcgaggcgcttcagggcgaggcggcagttgcgaaaagcgtgcgcgcgttaggctcatcagccattattgatcgcccgctggacgaccgtgccttggaaacgctccgccgccacttaatcgatccgccgagccatcgctcagaagtcgcgatctacggtatgggcggcaagattgcagcgaagggccgtgaagaaacggctttcgtacatcgcacggagcttatggccttcgagtaccgtaccgactgggacgaaccggccgacgacgagaagaatgtagcgtgggtgcgttcagtccgccaggagatggcggagcacacaactggtggtacctacaccaacactgtcgatttgggtgtagagaactggttatggggttattatgaggaaaatcttccccgtctgatgggcgtgaagcaccgttacgatccggatggggtgtttcagcatgcgcacagtgttcctctgagtcttactgaggccgaggcctcacgctttggaattccagagcaaattcgtcgtactctgcgtgcagcaggccgcttgacctga

SEQ ID NO: 8 - protein, 473 aa, Phytohabitans suffuscus
MSTAPVFGGRIVTRLDPSYEEARQVWNAVPDHRPLEIRYCRDAQDVAAALRDALARGLPFAARSGGHSAAALSVVPDGVVIDVSPLNSISLDQDASQVTFGSGATLGAIYRTLWESGVTVPAGTCPNIGVAGHVLGGGLGILSRSRGPLVDHLIGVTMVDARGELVTADEENHSDLLWACRGGGGGNYGIATSFTLRTQPIADVTLFNITWRFDDLGPAVKAWQSWLADADHRINPFMMLYPREQDMVATVGVMEGTPDAFEPLVKPLLDAVEPSEVVIEPMSFIQAVDTVEALQGEAAVAKSVRALGSSAIIDRPLDDRALETLRRHLIDPPSHRSEVAIYGMGGKIAAKGREETAFVHRTELMAFEYRTDWDEPADDEKNVAWVRSVRQEMAEHTTGGTYTNTVDLGVENWLWGYYEENLPRLMGVKHRYDPDGVFQHAHSVPLSLTEAEASRFGIPEQIRRTLRAAGRLT

SEQ ID NO: 9 - DNA, 1434 bp, Streptomyces sp.
  source 1..1434: mol_type = genomic DNA
atggcaaccccgtcggcattttcaggtagcgtcttaactcccggagacgacggctttgaggcagcgcaggtgacttggaatgcgtgttattccagccgcccccgtgaggttatggtgtgtcacgacgccgctagtgtagcagaagccgtgcgtagcgttcgtgaacgcggccttccgtttcgcgtgcgctctggcggtcacagtatgtgtggcttatccaatttagacgacggcgtcattatcgatttaggcggcctgggcggcgttgagcttaccccggaccgccagacggttcgcattggtggtggcgcccgtttggctgatgtctacaacacactgtgggaccatcgcctcacagttcctgcaggcacttgtccccgcatcggggtaggcggacatgtgctcggcggcggaatgggagttttgtcacgcagccgtggtgcccttgtagatcatctgaccgcgcttgagatggtggatgctgagggtcgtttattgcgcgtgagcgaggacgaaaatcctgaccttttctgggcctgtcgcggcggtggcggcggcaacttcggcatcgtgacggcctacgaactccgcccaacacccattgatgacgtgacgatctttactgttagctggacttggtcgcaacttccagatgctgtacgtgcctggcaacgctggctcggttctgcggagagccgtatcaactctttcctttctctcttccctcaacaacaggacatggttgtcgcgttcggtgtctttgacggccctgccgctgactttcgtccgctcctggcgccgcttactgccgaagtcgccccagaggcagaggtggttgaggaagtaccctttattcaagccgtagacacggtcgaggccttacaaggcgaagcggcggcagcggagcaagtccgcgcgcagggctcgtcagctattatcgcgaaccccttaaacgatgaggccctcgctaccttgcaggaattcctcaccgacccgccttcccaccgtgcggaagtagcggtttatggtatgggcggggtgatcggtgagcgtgagcgtggagatacggcttttgttcatcgtacgggcttgatggcctttgagtaccgtacggactgggatagtcccgaggacgatcgtttaaaccttgactgggtgacgcgtctgcgtcacgccatggccgagcacaccaccggcgctgcctacgtaaatacgatcgatttggccttagaaaactggttatgggcatattacgaggaaaatctgcctcgtttaatggctgttaagcgtcgctacgatccggagaatgtattccaccatccccattcaattccaggatcgctgacggcggaagcagcccgtgcccacggtgtccccgaagcgacacttaagcgcctgcacgatgacggactgttggacggtccattagactaa

SEQ ID NO: 10 - protein, 477 aa, Streptomyces sp.
MATPSAFSGSVLTPGDDGFEAAQVTWNACYSSRPREVMVCHDAASVAEAVRSVRERGLPFRVRSGGHSMCGLSNLDDGVIIDLGGLGGVELTPDRQTVRIGGGARLADVYNTLWDHRLTVPAGTCPRIGVGGHVLGGGMGVLSRSRGALVDHLTALEMVDAEGRLLRVSEDENPDLFWACRGGGGGNFGIVTAYELRPTPIDDVTIFTVSWTWSQLPDAVRAWQRWLGSAESRINSFLSLFPQQQDMVVAFGVFDGPAADFRPLLAPLTAEVAPEAEVVEEVPFIQAVDTVEALQGEAAAAEQVRAQGSSAIIANPLNDEALATLQEFLTDPPSHRAEVAVYGMGGVIGERERGDTAFVHRTGLMAFEYRTDWDSPEDDRLNLDWVTRLRHAMAEHTTGAAYVNTIDLALENWLWAYYEENLPRLMAVKRRYDPENVFHHPHSIPGSLTAEAARAHGVPEATLKRLHDDGLLDGPLD

SEQ ID NO: 11 - DNA, 1434 bp, Streptomyces varsoviensis
  source 1..1434: mol_type = genomic DNA
atgagtggagctacaagtgcacgttttgccggaacagttctggcccccggtgacccgggcttcgaagatgccagccgtacctggaacgcctgcttcgcaagccgccctcgtgaggttatggtgtgccattcgacggcagaggtcgccgatgcggtacgtgaggttcgcgaacgcggcgtgtcattccgcgtccgttccggtggtcactcaatggccgggttatcgtcggttgaggacggcgtggtcattgatctgggcggacttgacgcaatttcactttcggcggatcgttcacttgtcacggtgggtggcggtacgcgcttaggcgatatttaccgcgagttatggcgctctggtgtgactgttcctgccggcacctgtccgcgtatcggcattggtgggcatgttttaggtggcggtatgggtgttctctcccgtagccgtggcgcgctggtagatcacctggcggccctggagttcgttgacgctgaggggcgcgcatatcgtgtagacgagaatgagcatccggacctgttctgggcatgccgcggtggcggggggggttcctatggaatcgccacgtcctatactttgcgcacgagtcctgttgacgaagtgacccttttcactttgagctggaattgggaacgcttcgctcccgcagtccgtgcctggcaatcctggctcgcctcgactgacgaccgtgtgaatacatttcttagtctcttcccccgtcaacaagacctggtagcggcgttcggtgtttacgacggaccagcggcagaattcgaagagctgctgcgcccgcttgtggcagctgtacgtccccaagatcaagtagtggagcccatgccattcattcaagcagtggacacggttgagcttctgcagggcgaggcagcggctgccgatcaggtgcgcgcccaagggagcagtgcaattatcggcggaccactggacggcgccgcaatcgcgacacttcaacaattcctgacggatccgccctcacaccgtagcgaggtcgcggtatatggcatgggcggtgcgattgctcgtcctgcgcgtaccgacacagccttcgttcaccgtaccggtcttatggcatttgagtatcgtacagactgggaccgcagcgaggacgacgcgaagaacctcgcgtggatcaaggcccttcgtcatgccatggctccccatactacgggagccgcctatgtgaataccattgacttagctttagagaactggctgtgggcgtactatgaagaaaacttgccccgtcttattgaggtaaagcgtcgctacgacccgggagacgtgtttcatcatccgcacagcattcctttaacggtgaccgaagctgatatcgctcgctatggcattcccgaggctgttgctgcgcgtctccgtgccgcaggccttactgaccgccaataa

SEQ ID NO: 12 - protein, 477 aa, Streptomyces varsoviensis
MSGATSARFAGTVLAPGDPGFEDASRTWNACFASRPREVMVCHSTAEVADAVREVRERGVSFRVRSGGHSMAGLSSVEDGVVIDLGGLDAISLSADRSLVTVGGGTRLGDIYRELWRSGVTVPAGTCPRIGIGGHVLGGGMGVLSRSRGALVDHLAALEFVDAEGRAYRVDENEHPDLFWACRGGGGGSYGIATSYTLRTSPVDEVTLFTLSWNWERFAPAVRAWQSWLASTDDRVNTFLSLFPRQQDLVAAFGVYDGPAAEFEELLRPLVAAVRPQDQVVEPMPFIQAVDTVELLQGEAAAADQVRAQGSSAIIGGPLDGAAIATLQQFLTDPPSHRSEVAVYGMGGAIARPARTDTAFVHRTGLMAFEYRTDWDRSEDDAKNLAWIKALRHAMAPHTTGAAYVNTIDLALENWLWAYYEENLPRLIEVKRRYDPGDVFHHPHSIPLTVTEADIARYGIPEAVAARLRAAGLTDRQ

SEQ ID NO: 13 - DNA, 1518 bp, Actinomadura pelletieri
  source 1..1518: mol_type = genomic DNA
atgatcagtcgtcgctcttttgtcgctggtgtaggtggagccgcagcccttgcggcgacagcagttcctacgcccgctcatgcgcgcccagcctcgcgtatccgttgggatcgtttgcgtcgtcacttgcgcggtcgcttagtccttcctagcgatgcggactatccgaaggcgaagcaactgtaccagatggagttcgacgagattcatccgcaagcaattgcatactgtacctgcacttcagacgtgagcttgtgcctgcgttttgctcaggaccacgacctgcatctggcagtgcgtagcggtggtcacagccacggcggttattccaccacccccggtctggttattgacgtgtctcgcatgaattcggtggtccctggcgaacagtctacaagcatcggtacaggtgcccaaacggttgacatcacgaatgccttggccccggccggtttggcagtttcgacgggttactgccccactgtcgcggccggcggttttctccagggcggtggtgttggcctgttaacccgtgcaaccgggattgcaagcgacaaggttcgtagcgcccgtgttgtcttagcagacggccgtgtagtgaccgcgtccccctctcgccacgaagatttatattgggccctgcgcggtggtggcggcggtaatttcggcatcgtcaccagttatgaggttacgccaacgcctctcacgcacttggcaactgcaatcttgatctggcgttatgatcaagcacttgacatgttggatggctggaccaagtggttagaggatgctccttggacgattggctcgggattaaacgtacagcttgaagactccgcgccgggtaaggtgccagtagcaagcgtgcttgtggggagcaccgacaccggcccggggttcgcggcagaggtcgagcgtctggtaagcttagtaggtcacgcgcctacgttccgtcgcgatgaagtggagccctatcaaaagatcatgatgggcttattcgactgtgacgatttgaccgtagatgagtgccaccgcagtgacacgttcccctctggtcgtttaccacgtgcagcctttggcttcgagcgttcgcgtatgttcggccgcgcgatgcctcgcgacggctgggcaaagactttagcgctgttcgatacggagcgtcgcgcgcgtcaaacccaccagctgcaagtgacggcgctgggtggtcgtgccaatactctgtcccgtactgagacagcgtacgtacaccgcgacacgcgttttatggtaacgttatatggctggcatcgtgagggccctgtaagcgcggaagataaagcggtggctggccgttggatcgatgccgctttcaacgtgattgatccttactccaatggcgagacttatcaaaacttcatcgacccgcgcttaaccgattggcagcgctcatattacgcggaaaattatcgtcgcttaaaacgcattaaacaccgttatgaccctcataatgtgttccgttcaccacagagcatccgctaa

SEQ ID NO: 14 - protein, 505 aa, Actinomadura pelletieri
MISRRSFVAGVGGAAALAATAVPTPAHARPASRIRWDRLRRHLRGRLVLPSDADYPKAKQLYQMEFDEIHPQAIAYCTCTSDVSLCLRFAQDHDLHLAVRSGGHSHGGYSTTPGLVIDVSRMNSVVPGEQSTSIGTGAQTVDITNALAPAGLAVSTGYCPTVAAGGFLQGGGVGLLTRATGIASDKVRSARVVLADGRVVTASPSRHEDLYWALRGGGGGNFGIVTSYEVTPTPLTHLATAILIWRYDQALDMLDGWTKWLEDAPWTIGSGLNVQLEDSAPGKVPVASVLVGSTDTGPGFAAEVERLVSLVGHAPTFRRDEVEPYQKIMMGLFDCDDLTVDECHRSDTFPSGRLPRAAFGFERSRMFGRAMPRDGWAKTLALFDTERRARQTHQLQVTALGGRANTLSRTETAYVHRDTRFMVTLYGWHREGPVSAEDKAVAGRWIDAAFNVIDPYSNGETYQNFIDPRLTDWQRSYYAENYRRLKRIKHRYDPHNVFRSPQSIR

SEQ ID NO: 15 - DNA, 1521 bp, synthetic construct
  misc_feature 1..1521: note = Synthetic polymer
  source 1..1521: mol_type = other DNA
atgaccgcagatccgagcagcgaacgtagcgatatgaatgaagcagatgaagtgaacgaagttgatgaactgagcgaaaccggtcagaccagcggcaccaaaggtaaacgtccgtttacaggtcgtgttattggtccggcagatggtgaatttgatgaagcacgtcgtgtttggaatgaatgttttgcagcacgtccgaaagaaattgtttattgtgcagatacccgtgatgttgttcgtgcactgcgtgaagttcgtcagcgtggtggtccgtttcgtgttcgtagcggtggtcatagcgcaagcggtctgagcgttctggatgatggtacagtgctggatgttagtggcctggatgatattcaggttagcgaagatgcaagcaccgttaccgttggtagcggtgcacatctgggtgatatttttcgtgccctgtgggcacgtggtgttaccgttccggcaggttattgtccgaccattggtattgcaggtcatgttttaggtggtggtgcaggtattctggtgcgtagccgtggttttctgagcgatcatctggttgcactggaaatggttgatagcgaaggtcgtattgttgttgcagatcatgatagtcatcatgaactgctgtgggcaagccgtggtggtggcggtggtaattttggcattgcaaccagctttaccctgcgtacccagccgattggtgatgttaccagttttaccattgcatgggattgggatcgtggtgccgaagcaattaaagcatggcaagaatggctggcaaccgcagatggtcgcattgttaccatgtttcatgcaattccgcaggatcaggatatgttttatgcaagcggttgttttgaaggtgatgcagcagaactggaaccgctgattgcaccgctggttcatgcagttgaaccgaccgaaaaagttgcagaaaccatgccgtggattgaagcactgagcttttatgaaggcatgcagggtgaagccatgaaagcaaccagcgttcgtgcaaaaatcaatctgagctttgttaccgaaccgctgggtgatcgtgccgttgaagaaatcaaaaaagcactggcacaggcaccgagccatcgtgcaatggttgttctgtatggtttaggtggcgcagttgcagcaaaaggtcgtcgtgaaaccgcatttgttcatcgtgatgcaccggttgcgctgtggtatacaaccagctgggatgatgaagccgaagatgatctgaattttgcctggattcagaatctgcgtgcaagcgttgcagcacataccgaaggtcgcggtagctatgttaactatattgatctgaccgttgaacactggctgtgggattattatgaagaaaatctgcctcgtctgatggccgtgaaaaaacgttatgatccggaagatgtttttcgtcatccgcagagcattccggttagcctgaccgaagcagaagcagccgaactgggtattccgcctcatattgccgaagaactgcgtgccgcacgtcagctgcgttaa

SEQ ID NO: 16 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSASGLSVLDDGTVLDVSGLDDIQVSEDASTVTVGSGAHLGDIFRALWARGVTVPAGYCPTIGIAGHVLGGGAGILVRSRGFLSDHLVALEMVDSEGRIVVADHDSHHELLWASRGGGGGNFGIATSFTLRTQPIGDVTSFTIAWDWDRGAEAIKAWQEWLATADGRIVTMFHAIPQDQDMFYASGCFEGDAAELEPLIAPLVHAVEPTEKVAETMPWIEALSFYEGMQGEAMKATSVRAKINLSFVTEPLGDRAVEEIKKALAQAPSHRAMVVLYGLGGAVAAKGRRETAFVHRDAPVALWYTTSWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 17 - DNA, 1539 bp, synthetic construct
  misc_feature 1..1539: note = Synthetic polymer
  source 1..1539: mol_type = other DNA
atgaccgcagatccgagcagcgaacgtagcgatatgaatgaagcagatgaagtgaacgaagttgatgaactgagcgaaaccggtcagaccagcggcaccaaaggtaaacgtccgtttacaggtcgtgttattggtccggcagatggtgaatttgatgaagcacgtcgtagcaccattcagaatctgcgttttattagcgataccacaccgcgtccgaaagaaattgtttattgtgcagatacccgtgatgttgttcgtgcactgcgtgaagttcgtcagcgtggtggtccgtttcgtgttcgtagcggtggtcatagcgcaagcggtctgagcgttctggatgatggtacagtgctggatgttagtggcctggatgatattcaggttagcgaagatgcaagcaccgttaccgttggtagcggtgcacatctgggtgatatttttcgtgccctgtgggcacgtggtgttaccgttccggcaggttattgtccgaccattggtattgcaggtcatgttttaggtggtggtgcaggtattctggtgcgtagccgtggttttctgagcgatcatctggttgcactggaaatggttgatagcgaaggtcgtattgttgttgcagatcatgatagtcatcatgaactgctgtgggcaagccgtggtggtggcggtggtaattttggcattgcaaccagctttaccctgcgtacccagccgattggtgatgttaccagttttaccattgcatgggattgggatcgtggtgccgaagcaattaaagcatggcaagaatggctggcaaccgcagatggtcgcattgttaccatgtttcatgcaattccgcaggatcaggatatgttttatgcaagcggttgttttgaaggtgatgcagcagaactggaaccgctgattgcaccgctggttcatgcagttgaaccgaccgaaaaagttgcagaaaccatgccgtggattgaagcactgagcttttatgaaggcatgcagggtgaagccatgaaagcaaccagcgttcgtgcaaaaatcaatctgagctttgttaccgaaccgctgggtgatcgtgccgttgaagaaatcaaaaaagcactggcacaggcaccgagccatcgtgcaatggttgttctgtatggtttaggtggcgcagttgcagcaaaaggtcgtcgtgaaaccgcatttgttcatcgtgatgcaccggttgcgctgtggtatacaaccagctgggatgatgaagccgaagatgatctgaattttgcctggattcagaatttacgtgcaagcgttgcagcacataccgaaggtcgcggtagctatgttaactatattgatctgaccgttgaacactggctgtgggattattatgaagaaaatctgcctcgtctgatggccgtgaaaaaacgttatgatccggaagatgtttttcgtcatccgcagagcattccggttagcctgaccgaagcagaagcagccgaactgggtattccgcctcatattgccgaagaactgcgtgcagcacgtcagctgcgttaa

SEQ ID NO: 18 - protein, 512 aa, synthetic construct
  REGION 1..512: note = Synthetic polymer
MTADPSSERSDMNEADEVNEVDELSETGQTSGTKGKRPFTGRVIGPADGEFDEARRSTIQNLRFISDTTPRPKEIVYCADTRDVVRALREVRQRGGPFRVRSGGHSASGLSVLDDGTVLDVSGLDDIQVSEDASTVTVGSGAHLGDIFRALWARGVTVPAGYCPTIGIAGHVLGGGAGILVRSRGFLSDHLVALEMVDSEGRIVVADHDSHHELLWASRGGGGGNFGIATSFTLRTQPIGDVTSFTIAWDWDRGAEAIKAWQEWLATADGRIVTMFHAIPQDQDMFYASGCFEGDAAELEPLIAPLVHAVEPTEKVAETMPWIEALSFYEGMQGEAMKATSVRAKINLSFVTEPLGDRAVEEIKKALAQAPSHRAMVVLYGLGGAVAAKGRRETAFVHRDAPVALWYTTSWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 19 - DNA, 1560 bp, synthetic construct
  misc_feature 1..1560: note = Synthetic polymer
  source 1..1560: mol_type = other DNA
atgaccgcagatccgagcagcgaacgtagcgatatgaatgaagcagatgaagtgaacgaagttgatgaactgagcgaaaccggtcagaccagcggcaccaaaggtaaacgtccgtttacaggtcgtgttattggtccggcagatggtgaatttgatgaagcacgtcgtagcaccattcagaatctgcgttttattagcgataccacaccgcgtccgaaagaaattgtttattgtgcagatacccgtgatgttgttcgtgcactgcgtgaagttcgtcagcgtggtggtccgtttcgtgttcgtagcggtggtcatagcgcaagcggtctgagcgttctggatgatggtacagtgctggatgttagtggcctggatgatattcaggttagcgaagatgcaagcaccgttaccgttggtagcggtgcacatctgggtgatatttttcgtgccctgtgggcacgtggtgttaccgttccggcaggttattgtccgaccattggtattgcaggtcatgttttaggtggtggtgcaggtattctggtgcgtagccgtggttttctgagcgatcatctggttgcactggaaatggttgatagcgaaggtcgtattgttgttgcagatcatgatagtcatcatgaactgctgtgggcaagccgtggtggtggcggtggtaattttggcattgcaaccagctttaccctgcgtacccagccgattggtgatgttaccagttttaccattgcatgggattgggatcgtggtgccgaagcaattaaagcatggcaagaatggctggcaaccgcagatggtcgcattgttaccatgtttcatgcaattccgcaggatcaggatatgttttatgcaagcggttgttttgaaggtgatgcagcagaactggaaccgctgattgcaccgctggttcatgcagttgaaccgaccgaaaaagttgcagaaaccatgccgtggattgaaaccaccatcttttatagcggtgtcgtgaacttcaaaaaagagattctgctggaagcaagcgcaggtaaaaaaacccgtgcaaaaatcaatctgagctttgttaccgaaccgctgggtgatcgtgccgttgaagaaatcaaaaaagcactggcacaggcaccgagccatcgtgcaatggttgttctgtatggtttaggtggcgcagttgcagcaaaaggtcgtcgtgaaaccgcatttgttcatcgtgatgcaccggttgcgctgtggtatacaaccagctgggatgatgaagccgaagatgatctgaattttgcctggattcagaatttacgtgcaagcgttgcagcacataccgaaggtcgcggtagctatgttaactatattgatctgaccgttgaacactggctgtgggattattatgaagaaaatctgcctcgtctgatggccgtgaaaaaacgttatgatccggaagatgtttttcgtcatccgcagagcattccggttagcctgaccgaagcagaagcagccgaactgggtattccgcctcatattgccgaagaactgcgtgcagcacgtcagctgcgttaa

SEQ ID NO: 20 - protein, 519 aa, synthetic construct
  REGION 1..519: note = Synthetic polymer
MTADPSSERSDMNEADEVNEVDELSETGQTSGTKGKRPFTGRVIGPADGEFDEARRSTIQNLRFISDTTPRPKEIVYCADTRDVVRALREVRQRGGPFRVRSGGHSASGLSVLDDGTVLDVSGLDDIQVSEDASTVTVGSGAHLGDIFRALWARGVTVPAGYCPTIGIAGHVLGGGAGILVRSRGFLSDHLVALEMVDSEGRIVVADHDSHHELLWASRGGGGGNFGIATSFTLRTQPIGDVTSFTIAWDWDRGAEAIKAWQEWLATADGRIVTMFHAIPQDQDMFYASGCFEGDAAELEPLIAPLVHAVEPTEKVAETMPWIETTIFYSGVVNFKKEILLEASAGKKTRAKINLSFVTEPLGDRAVEEIKKALAQAPSHRAMVVLYGLGGAVAAKGRRETAFVHRDAPVALWYTTSWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 21 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccgcgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 22 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSASGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 23 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgagctttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 24 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTSFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 25 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgatgttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 26 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTMFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 27 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttccatgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 28 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFHAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 29 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgatgttccatgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 30 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTMFHAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 31 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctagcggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 32 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAASGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 33 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaggcatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 34 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVEGMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 35 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcgatggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 36 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAMVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 37 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattatggtatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 38 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALWYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 39 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattataccacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 40 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYTTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 41 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacaagctgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 42 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTSWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 43 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaattatatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 44 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 45 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaagattaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaatacaatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 46 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKINLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 47 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccatgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttccatgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaattatatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 48 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFHAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 49 - DNA, 1521 bp, synthetic construct
  misc_feature 1..1521: note = Synthetic polymer
  source 1..1521: mol_type = other DNA
atgacagctgacccatctagcgagcgctccgacatgaatgaagcagatgaagttaacgaagtggatgaactgtctgaaacgggtcagacgtctggaaccaaaggcaaacgtccttttacaggacgcgtgatcggtccagctgatggagaattcgatgaagcgcgtcgcgtgtggaatgaatgttttgctgcgcgcccaaaagaaatcgtttactgcgcggatacgcgcgacgtcgttcgcgcgttgcgcgaggttcgtcagcgcggcggtccttttcgtgttcgttctggcggccattccgcgtctggactctcagtgctggatgacgggacggttctggacgtcagcggtctggatgacattcaagttagtgaagacgcctccacggttacggttgggagtggcgcgcatctgggtgatattttccgtgccctgtgggcccgcggcgtaaccgtaccagctggcttttgccccgaaatcggtatcgcgggccatgtcctggggggcggcgcgggaatcctggtgcgcagtcgtggtttcttatccgaccacttagtggccctggaaatggttgattctgaaggccgcattgtcgttgccgaccatgatagtcatcatgaactgctgtgggcaagccgtggtggaggcggtggtaattttggtatcgcgacgagtttcaccctgcgcacgcaacctatcggcgatgttacgctgtttaccattgcgtgggactgggaccgtggcgccgaagcgatcaaagcctggcaggaatggcttgctactgcagatggtcgcattaatacgcttttcattgcatatccacaggaccaggacatgttcgccgctctgggctgtttcgaaggcgatgcggcggaattagaaccgctgattgcgccgctggtccatgcggtggaaccgaccgaaaaagttgccgaaactatgccatggatcgaagcgctttcatttgtcgaaaccatgcaaggagaagcgatgaaggccacttcagtccgcgcgaaggggaacctgagttttgtgaccgagccgctgggtgatcgtgccgtcgaagaaatcaaaaaggcgctcgcgcaagcgccgagccatcgcgcggaggtggtcctgtatggcttaggcggcgcagttgccgcaaaaggtcgtcgcgaaactgcatttgtacatcgcgacgctccagtggcattaaattatcacacagattgggacgacgaagcagaagatgacctgaattttgcgtggattcagaacctgcgcgcgagtgtggcagcacatacagaaggccgcggcagctatgtcaattatatcgatctgacggtagagcactggctgtgggattattatgaagaaaacttgccccggttaatggcagtaaaaaaacgctacgacccggaagatgtattccgtcacccgcagagcattccggtgtcattaactgaggcagaggcggcggaactcggaattcccccgcacattgccgaagagttacgcgcggcccgccagttgcgctaa

SEQ ID NO: 50 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSASGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 51 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCGCGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGATGTTCCATGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATACAATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 52 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSASGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTMFHAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 53 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGATGTTCCATGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATTATATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 54 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTMFHAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 55 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGATGGTGGTCCTGTATGGCTTAGGCGGCGCAGTTGCCGCAAAAGGTCGTCGCGAAACTGCATTTGTACATCGCGACGCTCCAGTGGCATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATTATATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 56 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAMVVLYGLGGAVAAKGRRETAFVHRDAPVALNYHTDWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 57 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTATGGTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATTATATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 58 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALWYHTDWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 59 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTAAATTATACCACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATTATATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 60 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYTTDWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 61 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTAAATTATCACACAAGCTGGGACGACGAAGCAGAAGATGACCTGAATTTTGCGTGGATTCAGAACCTGCGCGCGAGTGTGGCAGCACATACAGAAGGCCGCGGCAGCTATGTCAATTATATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 62 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTSWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 63 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTATTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATACAATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 64 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGYCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 65 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTGTGACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATACAATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 66 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRIVTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 67 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTTATGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATACAATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 68 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFYETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 69 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTAGCGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTTATGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATACAATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 70 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAASGCFEGDAAELEPLIAPLVHAVEPTEKVAETMPWIEALSFYETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 71 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCTATGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATACAATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 72 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGYGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 73 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGAATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATACAATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 74 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVELNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 75 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCTATGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGAATTAAATTATCACACAGATTGGGACGACGAAGCAGAAGATGACCTGAATTTTGCGTGGATTCAGAACCTGCGCGCGAGTGTGGCAGCACATACAGAAGGCCGCGGCAGCTATGTCAATACAATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 76 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGYGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVELNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 77 - protein, 1557 aa, synthetic construct
  REGION 1..1557: note = Synthetic polymer
ATGACAGCAGATCCTAGTAGTGAGCGCAGCGACATGAACGAGGCGGATGAGGTTAATGAGGTGGACGAGCTTTCTGAGACAGGGCAAACGAGCGGAACGAAGGGCAAGCGCCCGTTTACCGGGCGCGTTATCGGCCCGGCCGACGGAGAATTTGACGAGGCACGTCGCGTGTGGAACGAGTGTTTCGCAGCTCGTCCAAAAGAGATCGTTTATTGTGCGGACACGCGTGACGTGGTGCGCGCTCTGCGCGAGGTTCGCCAACGTGGTGGACCTTTCCGTGTACGTAGCGGTGGTCATTCTGCCAGCGGACTGTCCGTGCTGGACGATGGGACTGTCCTCGACGTGTCGGGACTTGACGACATCCAAGTCAGCGAGGACGCCTCGACTGTTACTGTTGGTAGCGGAGCCCATTTGGGCGACATCTTTCGCGCCTTGTGGGCCCGTGGTGTAACTGTGCCGGCCGGCTATTGTCCAACCATCGGTATCGCAGGTCATGTACTCGGCGGCGGGTACGGCATCTTGGTGCGCAGCCGCGGCTTTCTGTCAGACCACCTGGTTGCTCTTGAGATGGTTGATTCAGAAGGACGCATTGTCGTGGCCGACCACGACTCTCATCATGAGCTCCTGTGGGCATCACGTGGTGGTGGCGGTGGTAATTTTGGCATTGCGACTAGTTTTACACTGCGTACCCAGCCGATCGGTGACGTCACCAGTTTCACCATCGCCTGGGATTGGGACCGTGGCGCAGAAGCCATTAAGGCTTGGCAAGAGTGGTTAGCCACGGCGGACGGGCGCATTGTAACGATGTTCCATGCAATTCCTCAAGACCAGGATATGTTTTACGCGAGCGGTTGCTTTGAAGGAGATGCAGCAGAGCTTGAGCCACTCATCGCACCCTTGGTCCATGCAGTGGAGCCGACAGAGAAGGTTGCCGAGACTATGCCTTGGATCGAAGCACTTTCATTCTACTCTGGGGTTGTGAATTTCAATACGGCGAATTTTAAGAAGGAAATCCTGCTGGATCGTAGTGCCGGCAAGAAGACCCGTGCAAAGATCAATTTGTCATTTGTGACTGAGCCCTTGGGCGATCGCGCCGTTGAAGAGATTAAGAAGGCCCTTGCCCAAGCGCCCTCTCATCGTGCCATGGTGGTACTGTACGGCCTTGGTGGTGCTGTGGCAGCGAAGGGTCGCCGTGAGACGGCGTTTGTCCACCGCGACGCCCCAGTTGAATTGTGGTATACCACTTCCTGGGATGACGAGGCGGAAGACGACCTTAATTTCGCGTGGATTCAGAACCTGCGCGCTTCGGTCGCCGCGCATACAGAGGGCCGCGGTAGTTATGTTAACTACATCGATTTGACCGTTGAGCATTGGTTATGGGACTACTACGAGGAAAATCTGCCTCGTCTGATGGCAGTTAAGAAGCGTTACGATCCCGAGGACGTGTTCCGCCATCCCCAAAGCATCCCCGTGTCGTTAACCGAGGCCGAAGCAGCAGAACTTGGCATCCCACCTCACATCGCGGAAGAGCTCCGTGCAGCACGTCAACTTCGTTAA

SEQ ID NO: 78 - protein, 518 aa, synthetic construct
  REGION 1..518: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSASGLSVLDDGTVLDVSGLDDIQVSEDASTVTVGSGAHLGDIFRALWARGVTVPAGYCPTIGIAGHVLGGGYGILVRSRGFLSDHLVALEMVDSEGRIVVADHDSHHELLWASRGGGGGNFGIATSFTLRTQPIGDVTSFTIAWDWDRGAEAIKAWQEWLATADGRIVTMFHAIPQDQDMFYASGCFEGDAAELEPLIAPLVHAVEPTEKVAETMPWIEALSFYSGVVNFNTANFKKEILLDRSAGKKTRAKINLSFVTEPLGDRAVEEIKKALAQAPSHRAMVVLYGLGGAVAAKGRRETAFVHRDAPVELWYTTSWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 79 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTATGGTATACCACAAGCTGGGACGACGAAGCAGAAGATGACCTGAATTTTGCGTGGATTCAGAACCTGCGCGCGAGTGTGGCAGCACATACAGAAGGCCGCGGCAGCTATGTCAATACAATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 80 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALWYTTSWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 81 - protein, 1521 aa, synthetic construct
  REGION 1..1521: note = Synthetic polymer
ATGACAGCTGACCCATCTAGCGAGCGCTCCGACATGAATGAAGCAGATGAAGTTAACGAAGTGGATGAACTGTCTGAAACGGGTCAGACGTCTGGAACCAAAGGCAAACGTCCTTTTACAGGACGCGTGATCGGTCCAGCTGATGGAGAATTCGATGAAGCGCGTCGCGTGTGGAATGAATGTTTTGCTGCGCGCCCAAAAGAAATCGTTTACTGCGCGGATACGCGCGACGTCGTTCGCGCGTTGCGCGAGGTTCGTCAGCGCGGCGGTCCTTTTCGTGTTCGTTCTGGCGGCCATTCCATGTCTGGACTCTCAGTGCTGGATGACGGGACGGTTCTGGACGTCAGCGGTCTGGATGACATTCAAGTTAGTGAAGACGCCTCCACGGTTACGGTTGGGAGTGGCGCGCATCTGGGTGATATTTTCCGTGCCCTGTGGGCCCGCGGCGTAACCGTACCAGCTGGCTTTTGCCCCGAAATCGGTATCGCGGGCCATGTCCTGGGGGGCGGCGCGGGAATCCTGGTGCGCAGTCGTGGTTTCTTATCCGACCACTTAGTGGCCCTGGAAATGGTTGATTCTGAAGGCCGCATTGTCGTTGCCGACCATGATAGTCATCATGAACTGCTGTGGGCAAGCCGTGGTGGAGGCGGTGGTAATTTTGGTATCGCGACGAGTTTCACCCTGCGCACGCAACCTATCGGCGATGTTACGCTGTTTACCATTGCGTGGGACTGGGACCGTGGCGCCGAAGCGATCAAAGCCTGGCAGGAATGGCTTGCTACTGCAGATGGTCGCATTAATACGCTTTTCATTGCATATCCACAGGACCAGGACATGTTCGCCGCTCTGGGCTGTTTCGAAGGCGATGCGGCGGAATTAGAACCGCTGATTGCGCCGCTGGTCCATGCGGTGGAACCGACCGAAAAAGTTGCCGAAACTATGCCATGGATCGAAGCGCTTTCATTTGTCGAAACCATGCAAGGAGAAGCGATGAAGGCCACTTCAGTCCGCGCGAAGGGGAACCTGAGTTTTGTGACCGAGCCGCTGGGTGATCGTGCCGTCGAAGAAATCAAAAAGGCGCTCGCGCAAGCGCCGAGCCATCGCGCGGAGGTGGTCCTGTATGGCTTAGGCGGCGCAGTTGCCGCAAAAGGTCGTCGCGAAACTGCATTTGTACATCGCGACGCTCCAGTGGCATTATGGTATACCACAAGCTGGGACGACGAAGCAGAAGATGACCTGAATTTTGCGTGGATTCAGAACCTGCGCGCGAGTGTGGCAGCACATACAGAAGGCCGCGGCAGCTATGTCAATTATATCGATCTGACGGTAGAGCACTGGCTGTGGGATTATTATGAAGAAAACTTGCCCCGGTTAATGGCAGTAAAAAAACGCTACGACCCGGAAGATGTATTCCGTCACCCGCAGAGCATTCCGGTGTCATTAACTGAGGCAGAGGCGGCGGAACTCGGAATTCCCCCGCACATTGCCGAAGAGTTACGCGCGGCCCGCCAGTTGCGCTAA

SEQ ID NO: 82 - protein, 506 aa, synthetic construct
  REGION 1..506: note = Synthetic polymer
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALWYTTSWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR